(19) United States
(12) Patent Application Publication
(10) Pub. No.: US 2016/0248020 A1
(43) Pub. Date: Aug. 25, 2016

(54) COMPOSITIONS CONTAINING FLUORENE SUBSTITUTED TRIAZINE DERIVED COMPOUNDS, AND ELECTRONIC DEVICES CONTAINING THE SAME

(71) Applicants: DOW GLOBAL TECHNOLOGIES LLC, Midland, MI (US); ROHM AND HAAS ELECTRONIC MATERIALS KOREA LTD., Cheonan-Si (KR)

(72) Inventors: Mark E. Ondari, Midland, MI (US); Kyoung Moo Koh, Midland, MI (US); Dean M. Welsh, Midland, MI (US); Tomas D. Paine, Bay City, MI (US); Liang Hong, Schwenksville, PA (US); Kenneth E. Stockman, Midland, MI (US); Hong-Yeop Na, Seoul (KR); Kyung-Joo Lee, Seoul (KR)

(21) Appl. No.: 14/916,014

(22) PCT Filed: Nov. 10, 2014
(86) PCT No.: PCT/US2014/064723

§ 371 Date: Mar. 2, 2016

Related U.S. Application Data
(60) Provisional application No. 61/902,946, filed on Nov. 12, 2013.

(51) Int. Cl. H01L 51/00; C07D 251/24

(52) U.S. Cl. CPC H01L 51/0067; C07D 251/24; H01L 51/0058; H01L 51/0054; H01L 51/5016

(57) ABSTRACT

The invention provides a composition comprising at least one fluorene substituted triazine derived compound of Formula 1, as described herein, and electronic devices containing the same. Such devices have improved efficiency and better driving voltage.

### REFERENCE TO RELATED APPLICATIONS

The present application claims the benefit of U.S. Provisional Application No. 61/902,946, filed Nov. 12, 2013, and incorporated herein by reference.

### BACKGROUND

Electroluminescence (EL) devices are display devices that employ stacks of films containing organic aromatic compounds as an electroluminescent layer. See, for example, devices and/or triazine compounds described in the following references: U.S. Publications 2012/0286249, 2011/0095282, US2010/0039026, US2006/0251919; U.S. Pat. No. 7,807,276; International Publications WO 2007/023840, WO 2005/105950; Zong et al., New Optoelectronic Materials Based on Bitriazines: Synthesis and Properties, Organic Letter, vol. 10, 709-712; Zong et al., New Conjugated Triazine Based Molecular Materials for Application in Optoelectronic Devices: Design, Synthesis, and Properties, J. of Phys. Chem., 115, 2423-2427. Such aromatic compounds are generally classified as electroluminescent materials and charge transport materials. Several properties required for such electroluminescent and charge transport compounds, include high fluorescent quantum yield in solid state, high mobility of electrons and holes, chemical stability during vapor-deposition in vacuum, and the ability to form stable films. These desired features increase the lifetime of an EL device. However, there is a continual need for improved electroluminescent compounds and films containing the same.

### SUMMARY OF INVENTION

The invention provides a composition comprising at least one compound selected from Formula 1:

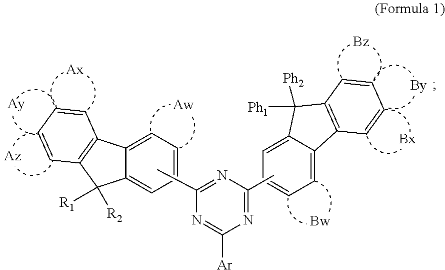

wherein, A is (CH), and x, y, w, z are each independently 0 or 4; and
wherein x=4, and y=0, z=0 or 4, and w=0 or 4, or
wherein y=4, and x=0, z=0, and w=0 or 4, or
wherein z=4, and x=0 or 4, y=0, and w=0 or 4, or
wherein w=4, and x=0, y=0, z=0; and

wherein, B is (CH), and x, y, w, z are each independently 0 or 4; and
wherein x=4, and y=0, z=0 or 4, and w=0 or 4, or
wherein y=4, and x=0, z=0, and w=0 or 4, or
wherein z=4, and x=0 or 4, y=0, and w=0 or 4, or
wherein w=4, and x=0, y=0, z=0; and

wherein R₁ and R₂ are each independently a C1-C20 alkyl or a C6-C30 aryl, each with or without one or more substituents; and

wherein Ph₁ and Ph₂ are each, independently, phenyl or a substituted phenyl; and

wherein Ar is an aryl, a substituted aryl, a heteroaryl, or a substituted heteroaryl; and wherein Ar comprises less than, or equal to, 16 carbons; and

wherein one or more hydrogens may be optionally substituted with deuterium.

### BRIEF DESCRIPTIONS OF THE FIGURES

FIG. 1 depicts a DSC profile of Compound 1.

FIG. 2 depicts a DSC profile of Compound 2.

FIG. 3 depicts a DSC profile of Compound 3.

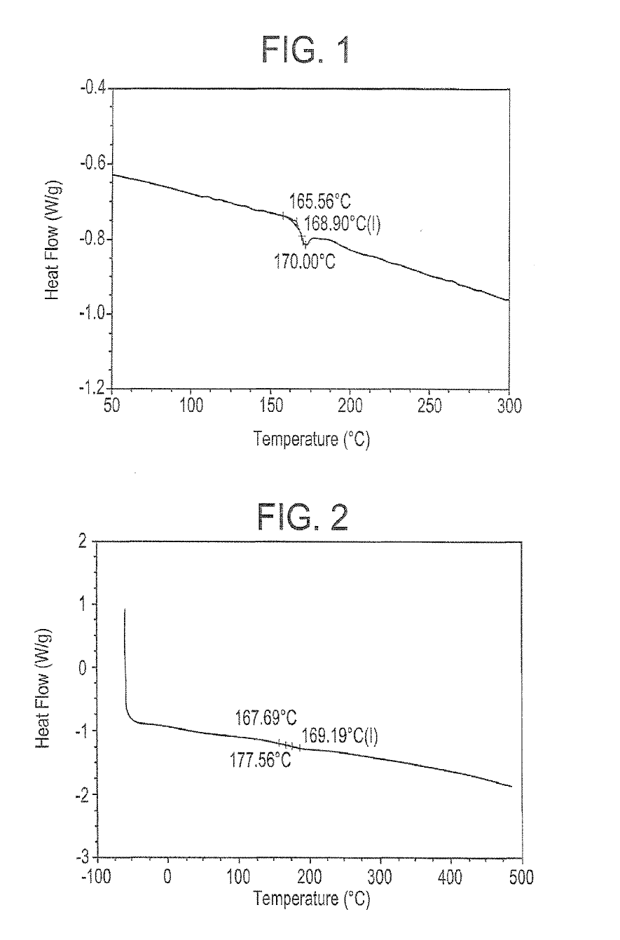

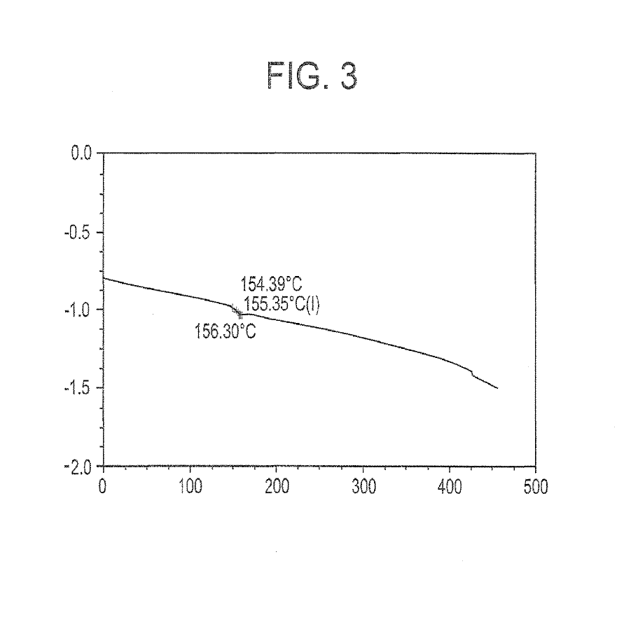

### DETAILED DESCRIPTION

As discussed above, the invention provides a composition comprising at least one compound selected from Formula 1:

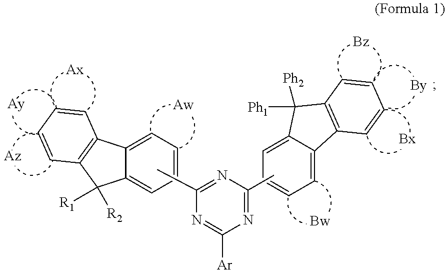

wherein, A is (CH), and x, y, w, z are each independently 0 or 4; and
wherein x=4, and y=0, z=0 or 4, and w=0 or 4, or
wherein y=4, and x=0, z=0, and w=0 or 4, or
wherein z=4, and x=0 or 4, y=0, and w=0 or 4, or
wherein w=4, and x=0, y=0, z=0; and

wherein, B is (CH), and x, y, w, z are each independently 0 or 4; and
wherein x=4, and y=0, z=0 or 4, and w=0 or 4, or
wherein y=4, and x=0, z=0, and w=0 or 4, or
wherein z=4, and x=0 or 4, y=0, and w=0 or 4, or
wherein w=4, and x=0, y=0, z=0; and

wherein R1 and R2 are each independently a C1-C20 alkyl or a C6-C30 aryl, each with or without one or more substituents; and

wherein Ph₁ and Ph₂ are each, independently, phenyl or a substituted phenyl; and

wherein Ar is an aryl, a substituted aryl, a heteroaryl, or a substituted heteroaryl; and wherein Ar comprises less than, or equal to, 16 carbons; and

wherein one or more hydrogens may be optionally substituted with deuterium.

An inventive compound may comprise a combination of two or more embodiments as described herein.

An inventive composition may comprise a combination of two or more embodiments as described herein.

In one embodiment, the Ar group comprises from 6 to 16 carbons, further from 7 to 16 carbons, further from 8 to 16 carbons.

In one embodiment, at least one compound is selected from Formula 2:

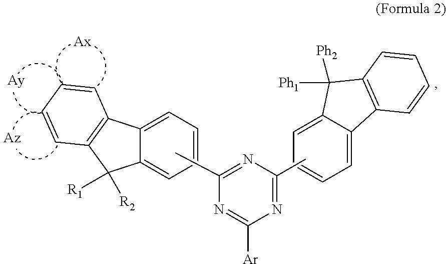

where the chemical groups are defined above for Formula 1. In a further embodiment, for Formula 2, x=0, y=0, and z=0 (or only hydrogen atoms on the noted portion of the ring).

In one embodiment, the Ar group of the inventive compound (Formula 1 or Formula 2) is selected from the following groups:

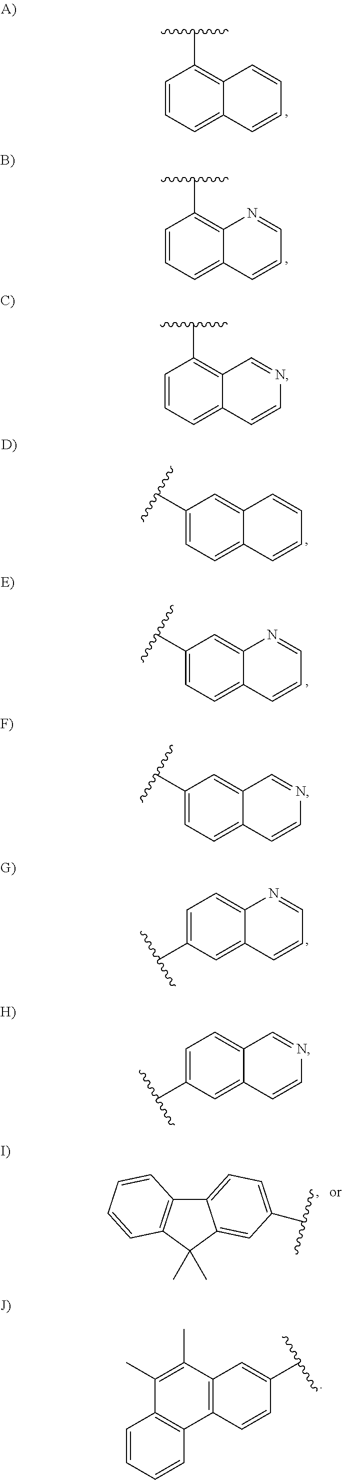

In one embodiment, the Ar group of the inventive compound (Formula 1 or Formula 2) is selected from groups A, D, I or J, each as shown above.

In one embodiment, the Ar group of the inventive compound (Formula 1 or Formula 2) is selected from groups D, I or J, each as shown above.

In one embodiment, the Ar group of the inventive compound (Formula 1 or Formula 2) is selected from groups A, D or I, each as shown above.

In the structures above, the external connection point of each substituent is indicated by a wavy line, as recommended by current IUPAC standards: Pure Appl. Chem., 2008, 80, 277 (Graphical representation standards for chemical structural diagrams).

In one embodiment, the Ar group of the inventive compound (Formula 1 or Formula 2) comprises from 8 to 16 carbon atoms, further from 9 to 16 carbons, further from 10 to 16 carbons. This range of carbon atoms is believed to provide better stacking of the compound in an electronic device. The improved stacking will improve the charge transport and efficiency of the device.

In one embodiment, the Ar group of the inventive compound (Formula 1 or Formula 2) comprises from 11 to 16 carbon atoms, further from 12 to 16 carbons. This range of carbon atoms is believed to provide even better stacking of the compound in an electronic device. The improved stacking will improve the charge transport and efficiency of the device.

In one embodiment, the Ar group of the inventive compound (Formula 1 or Formula 2) has a molecular weight from 120 to 290 g/mole, further from 120 to 280 g/mole.

In one embodiment, the compound (Formula 1 or Formula 2) has a glass transition temperature (Tg) greater than, or equal to, 150° C., further greater than, or equal to, 155° C., further greater than, or equal to, 160° C., further greater than, or equal to, 165° C., as determined by DSC.

In one embodiment, the compound (Formula 1 or Formula 2) has a Tg from 150° C. to 200° C., further from 155° C. to 200° C., further from 160° C. to 200° C., further from 165° C. to 200° C., as determined by DSC. High Tg values (≧150° C., more preferably ≧155° C., more preferably ≧160° C.) are desirable to improve the thermo stability of an electronic device.

In one embodiment, the compound (Formula 1 or Formula 2) has a molecular weight greater than, or equal to 620 g/mole. If the molecular weight is too low (for example, less than 620 g/mole), the Tg decreases to unacceptable levels.

In one embodiment, the compound (Formula 1 or Formula 2) has a molecular weight from 620 to 1000 g/mole, further from 650 to 1000 g/mole, further from 700 to 1000 g/mole.

In one embodiment, the compound (Formula 1 or Formula 2) has a Triplet Energy from 1.5 eV to 3.0 eV, further from 1.7 eV to 2.9 eV, further from 2.0 eV to 2.5 eV.

In one embodiment, the compound (Formula 1 or Formula 2) has a LUMO level from −1.5 eV to −2.5 eV, further from −1.6 eV to −2.2 eV, further from −1.7 eV to −2.0 eV.

In one embodiment, the compound (Formula 1 or Formula 2) has a HOMO level from −4.5 to −7.0 eV, further from −5.0 to −6.5 eV, further from −5.4 to −6.0 eV.

In one embodiment for the inventive compound (Formula 1 or Formula 2), R₁ and R₂ are each independently an alkyl.

In one embodiment for the inventive compound (Formula 1 or Formula 2), R₁ and R₂ are each independently a C1-C4 alkyl, further a C1-C3 alkyl, further a C1-C2 alkyl, further a C1 alkyl (R₁═R1, R₂═R2, etc.). The term “alkyl” is known in the art, and an alkyl is derived from an aliphatic hydrocarbon by deleting one hydrogen atom therefrom.

In one embodiment for the inventive compound (Formula 1 or Formula 2), R₁ and R₂ are each independently a propyl, ethyl or methyl, further ethyl or methyl, and further methyl.

In one embodiment for the inventive compound (Formula 1 or Formula 2), Ph₁ and Ph₂ are each a phenyl group (Ph₁=Ph1, and Ph₂=Ph2).

In one embodiment for the inventive compound (Formula 1 or Formula 2), for Ax, x=0; for Ay, y=0; for Az, z=0. In a further embodiment, for Aw, w=0.

In one embodiment for the inventive compound (Formula 1 or Formula 2), for Bx, x=0; for By, y=0; for Bz, z=0. In a further embodiment, for Bw, w=0.

In one embodiment, the compound (Formula 1 or Formula 2) is selected from the following:

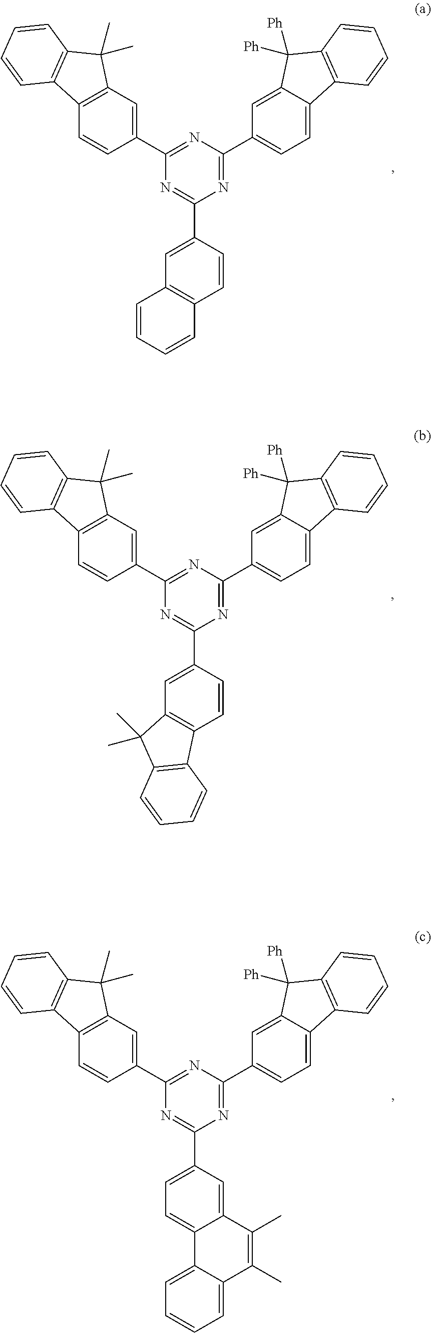 [SMILES: CC1(C)c2ccccc2-c2ccc(-c3nc(-c4ccc5c(c4)C(c4ccccc4)(c4ccccc4)c4ccccc4-5)nc(-c4ccc5ccccc5c4)n3)cc21 ; CC1(C)c2ccccc2-c2ccc(-c3nc(-c4ccc5c(c4)C(C)(C)c4ccccc4-5)nc(-c4ccc5c(c4)C(c4ccccc4)(c4ccccc4)c4ccccc4-5)n3)cc21 ; Cc1c(C)c2cc(-c3nc(-c4ccc5c(c4)C(C)(C)c4ccccc4-5)nc(-c4ccc5c(c4)C(c4ccccc4)(c4ccccc4)c4ccccc4-5)n3)ccc2c2ccccc12]

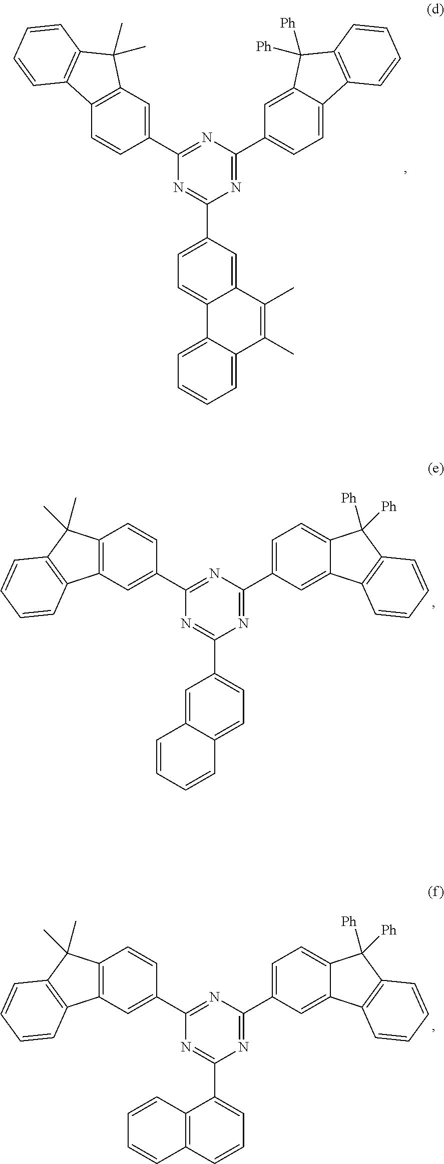 [SMILES: Cc1c(C)c2cc(-c3nc(-c4ccc5c(c4)C(C)(C)c4ccccc4-5)nc(-c4ccc5c(c4)C(c4ccccc4)(c4ccccc4)c4ccccc4-5)n3)ccc2c2ccccc12 ; CC1(C)c2ccccc2-c2cc(-c3nc(-c4ccc5c(c4)-c4ccccc4C5(c4ccccc4)c4ccccc4)nc(-c4ccc5ccccc5c4)n3)ccc21 ; CC1(C)c2ccccc2-c2cc(-c3nc(-c4ccc5c(c4)-c4ccccc4C5(c4ccccc4)c4ccccc4)nc(-c4cccc5ccccc45)n3)ccc21]

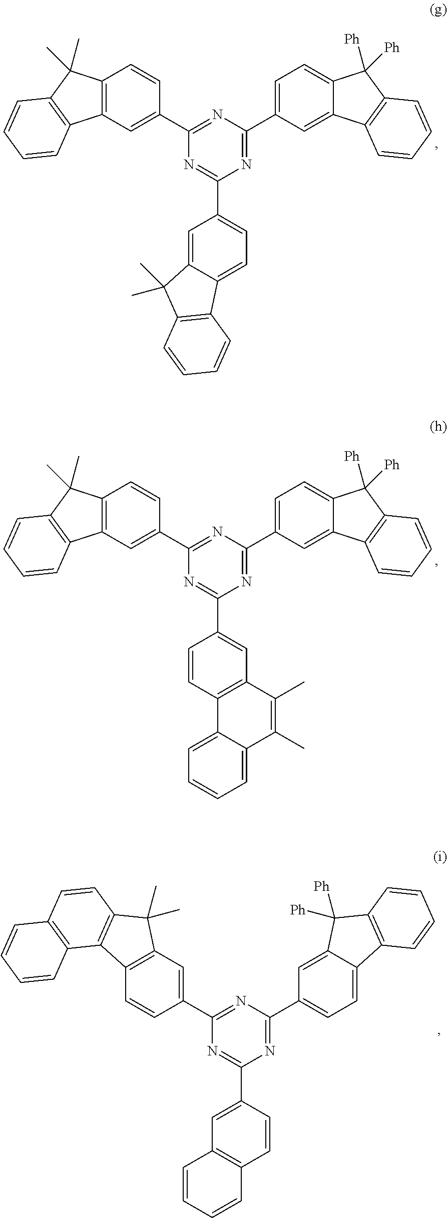 [SMILES: CC1(C)c2ccccc2-c2cc(-c3nc(-c4ccc5c(c4)-c4ccccc4C5(c4ccccc4)c4ccccc4)nc(-c4ccc5c(c4)C(C)(C)c4ccccc4-5)n3)ccc21 ; Cc1c(C)c2cc(-c3nc(-c4ccc5c(c4)-c4ccccc4C5(C)C)nc(-c4ccc5c(c4)-c4ccccc4C5(c4ccccc4)c4ccccc4)n3)ccc2c2ccccc12 ; CC1(C)c2cc(-c3nc(-c4ccc5c(c4)C(c4ccccc4)(c4ccccc4)c4ccccc4-5)nc(-c4ccc5ccccc5c4)n3)ccc2-c2c1ccc1ccccc21]

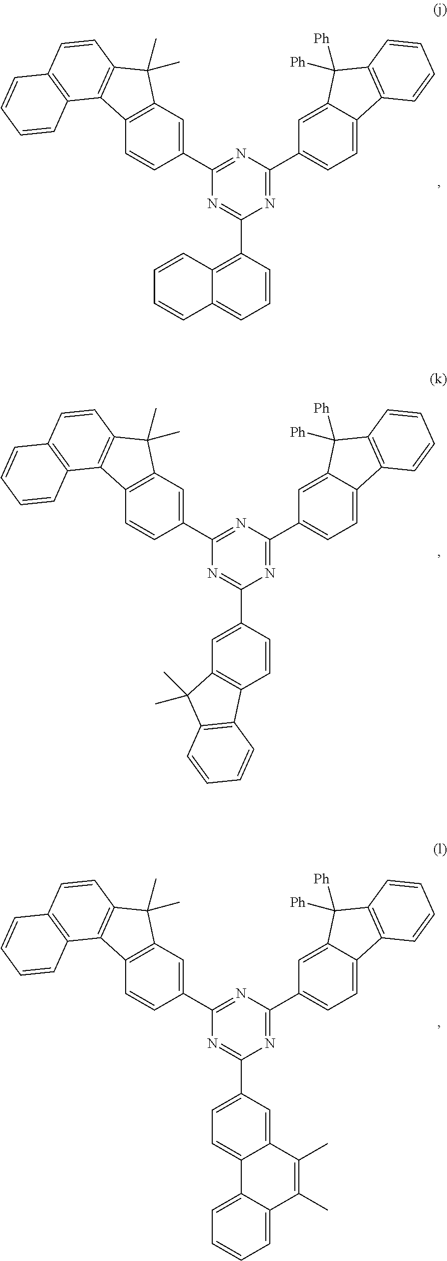 [SMILES: CC1(C)c2cc(-c3nc(-c4ccc5c(c4)C(c4ccccc4)(c4ccccc4)c4ccccc4-5)nc(-c4cccc5ccccc45)n3)ccc2-c2c1ccc1ccccc21 ; CC1(C)c2ccccc2-c2ccc(-c3nc(-c4ccc5c(c4)C(C)(C)c4ccc6ccccc6c4-5)nc(-c4ccc5c(c4)C(c4ccccc4)(c4ccccc4)c4ccccc4-5)n3)cc21 ; Cc1c(C)c2cc(-c3nc(-c4ccc5c(c4)C(C)(C)c4ccc6ccccc6c4-5)nc(-c4ccc5c(c4)C(c4ccccc4)(c4ccccc4)c4ccccc4-5)n3)ccc2c2ccccc12]

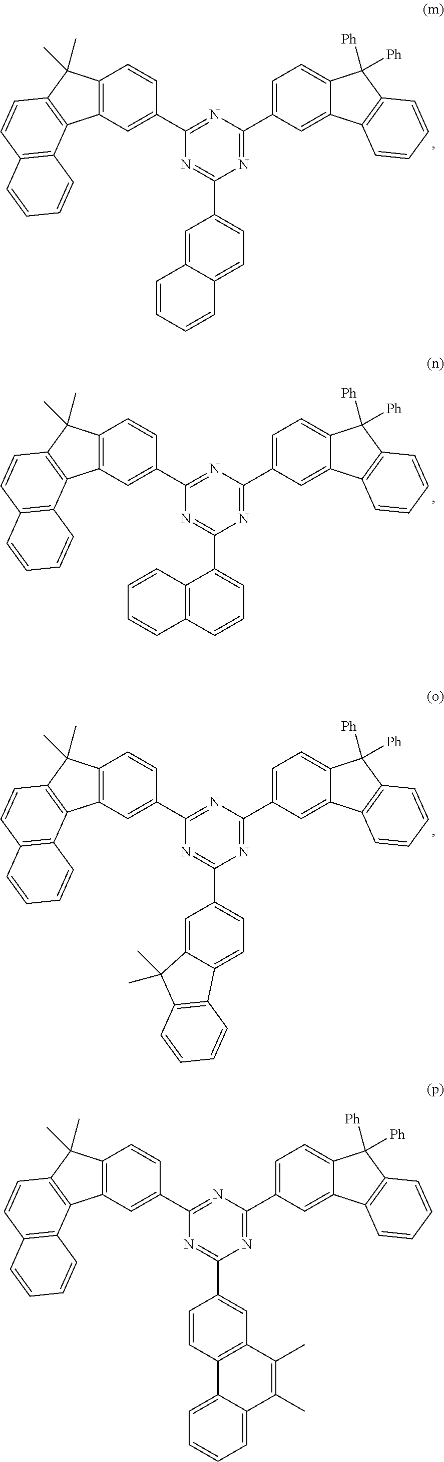 [SMILES: CC1(C)c2ccc(-c3nc(-c4ccc5c(c4)-c4ccccc4C5(c4ccccc4)c4ccccc4)nc(-c4ccc5ccccc5c4)n3)cc2-c2c1ccc1ccccc21 ; CC1(C)c2ccc(-c3nc(-c4ccc5c(c4)-c4ccccc4C5(c4ccccc4)c4ccccc4)nc(-c4cccc5ccccc45)n3)cc2-c2c1ccc1ccccc21 ; CC1(C)c2ccccc2-c2ccc(-c3nc(-c4ccc5c(c4)-c4c(ccc6ccccc46)C5(C)C)nc(-c4ccc5c(c4)-c4ccccc4C5(c4ccccc4)c4ccccc4)n3)cc21 ; Cc1c(C)c2cc(-c3nc(-c4ccc5c(c4)-c4c(ccc6ccccc46)C5(C)C)nc(-c4ccc5c(c4)-c4ccccc4C5(c4ccccc4)c4ccccc4)n3)ccc2c2ccccc12]

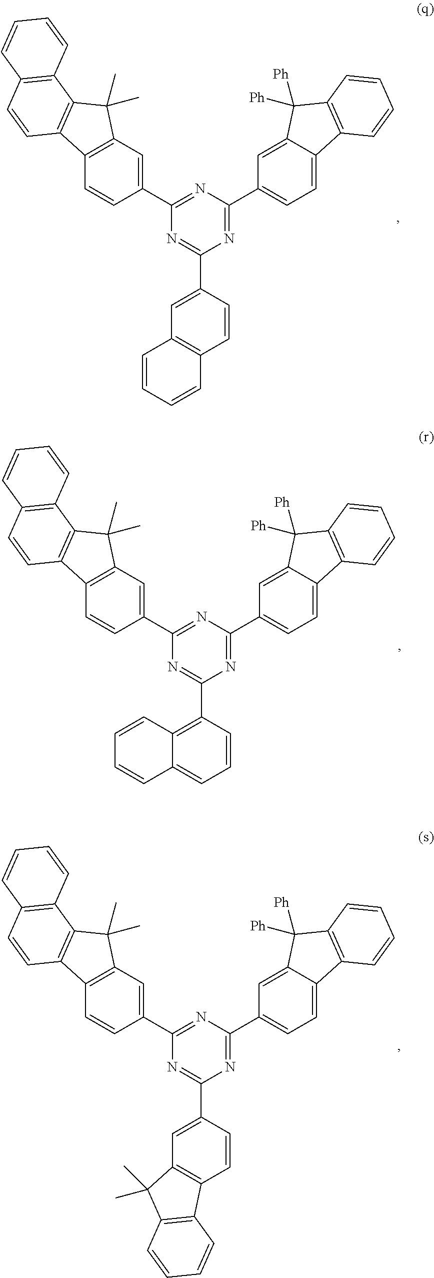 [SMILES: CC1(C)c2cc(-c3nc(-c4ccc5c(c4)C(c4ccccc4)(c4ccccc4)c4ccccc4-5)nc(-c4ccc5ccccc5c4)n3)ccc2-c2ccc3ccccc3c21 ; CC1(C)c2cc(-c3nc(-c4ccc5c(c4)C(c4ccccc4)(c4ccccc4)c4ccccc4-5)nc(-c4cccc5ccccc45)n3)ccc2-c2ccc3ccccc3c21 ; CC1(C)c2ccccc2-c2ccc(-c3nc(-c4ccc5c(c4)C(C)(C)c4c-5ccc5ccccc45)nc(-c4ccc5c(c4)C(c4ccccc4)(c4ccccc4)c4ccccc4-5)n3)cc21]

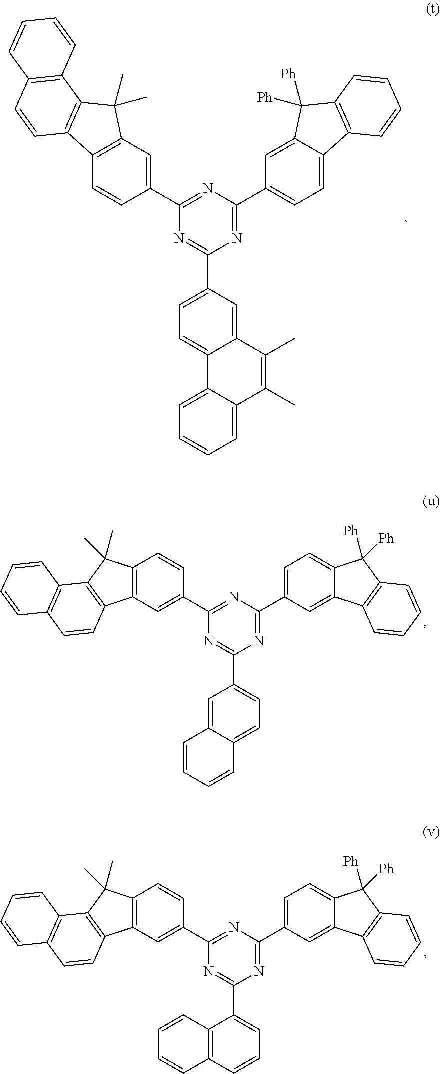 [SMILES: Cc1c(C)c2cc(-c3nc(-c4ccc5c(c4)C(C)(C)c4c-5ccc5ccccc45)nc(-c4ccc5c(c4)C(c4ccccc4)(c4ccccc4)c4ccccc4-5)n3)ccc2c2ccccc12 ; CC1(C)c2ccc(-c3nc(-c4ccc5c(c4)-c4ccccc4C5(c4ccccc4)c4ccccc4)nc(-c4ccc5ccccc5c4)n3)cc2-c2ccc3ccccc3c21 ; CC1(C)c2ccc(-c3nc(-c4ccc5c(c4)-c4ccccc4C5(c4ccccc4)c4ccccc4)nc(-c4cccc5ccccc45)n3)cc2-c2ccc3ccccc3c21]

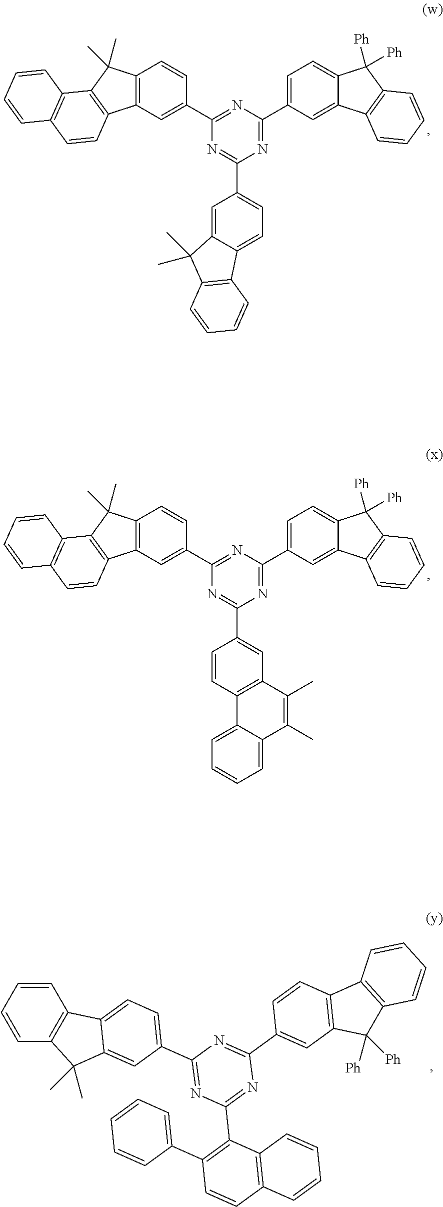 [SMILES: CC1(C)c2ccccc2-c2ccc(-c3nc(-c4ccc5c(c4)-c4ccc6ccccc6c4C5(C)C)nc(-c4ccc5c(c4)-c4ccccc4C5(c4ccccc4)c4ccccc4)n3)cc21 ; Cc1c(C)c2cc(-c3nc(-c4ccc5c(c4)-c4ccc6ccccc6c4C5(C)C)nc(-c4ccc5c(c4)-c4ccccc4C5(c4ccccc4)c4ccccc4)n3)ccc2c2ccccc12 ; CC1(C)c2ccccc2-c2ccc(-c3nc(-c4ccc5c(c4)C(c4ccccc4)(c4ccccc4)c4ccccc4-5)nc(-c4c(-c5ccccc5)ccc5ccccc45)n3)cc21]

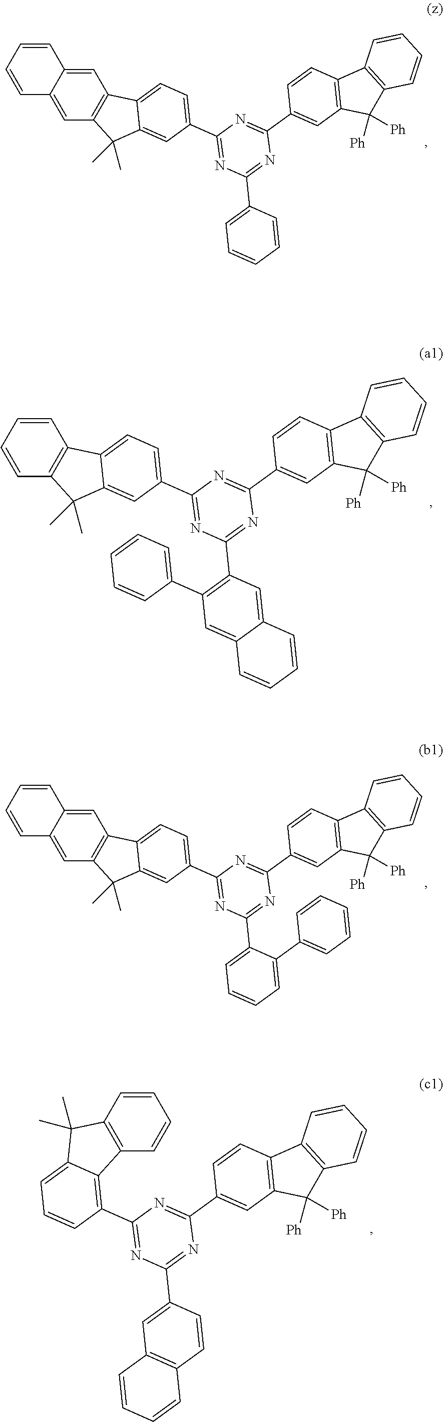 [SMILES: CC1(C)c2cc(-c3nc(-c4ccccc4)nc(-c4ccc5c(c4)C(c4ccccc4)(c4ccccc4)c4ccccc4-5)n3)ccc2-c2cc3ccccc3cc21 ; CC1(C)c2ccccc2-c2ccc(-c3nc(-c4ccc5c(c4)C(c4ccccc4)(c4ccccc4)c4ccccc4-5)nc(-c4cc5ccccc5cc4-c4ccccc4)n3)cc21 ; CC1(C)c2cc(-c3nc(-c4ccc5c(c4)C(c4ccccc4)(c4ccccc4)c4ccccc4-5)nc(-c4ccccc4-c4ccccc4)n3)ccc2-c2cc3ccccc3cc21 ; CC1(C)c2ccccc2-c2c(-c3nc(-c4ccc5c(c4)C(c4ccccc4)(c4ccccc4)c4ccccc4-5)nc(-c4ccc5ccccc5c4)n3)cccc21]

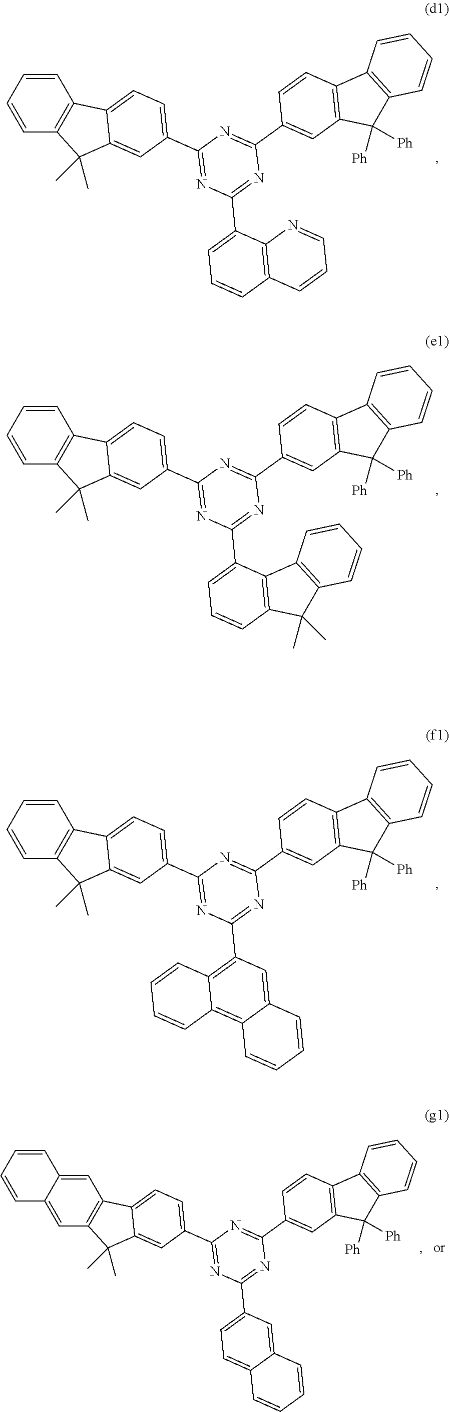 [SMILES: CC1(C)c2ccccc2-c2ccc(-c3nc(-c4ccc5c(c4)C(c4ccccc4)(c4ccccc4)c4ccccc4-5)nc(-c4cccc5cccnc45)n3)cc21 ; CC1(C)c2ccccc2-c2ccc(-c3nc(-c4ccc5c(c4)C(c4ccccc4)(c4ccccc4)c4ccccc4-5)nc(-c4cccc5c4-c4ccccc4C5(C)C)n3)cc21 ; CC1(C)c2ccccc2-c2ccc(-c3nc(-c4ccc5c(c4)C(c4ccccc4)(c4ccccc4)c4ccccc4-5)nc(-c4cc5ccccc5c5ccccc45)n3)cc21 ; CC1(C)c2cc(-c3nc(-c4ccc5c(c4)C(c4ccccc4)(c4ccccc4)c4ccccc4-5)nc(-c4ccc5ccccc5c4)n3)ccc2-c2cc3ccccc3cc21]

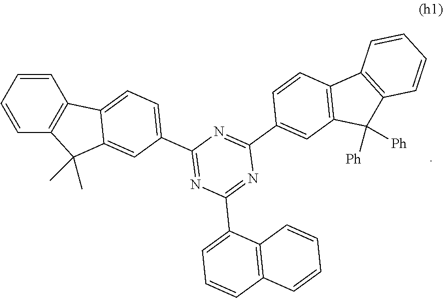 [SMILES: CC1(C)c2ccccc2-c2ccc(-c3nc(-c4ccc5c(c4)C(c4ccccc4)(c4ccccc4)c4ccccc4-5)nc(-c4cccc5ccccc45)n3)cc21]

In one embodiment, the inventive compound (Formula 1 or Formula 2) is selected from structures a through x, e1, g1 or h1, each as shown above.

In one embodiment, the inventive compound (Formula 1 or Formula 2) is selected from structures a, b, e through g, i through k, m through o, q through s or u through w, each as shown above.

In one embodiment, the compound (Formula 1 or Formula 2) comprises at least one deuterium atom.

In one embodiment, the compound (Formula 1 or Formula 2) does not comprise a deuterium atom.

In one embodiment, the compound (Formula 1 or Formula 2) has a purity greater than 99 percent.

In one embodiment, the composition comprises at least two compounds selected from Formula 1 or Formula 2.

In one embodiment, the composition comprises from 10 to 90 weight percent of the at least one compound (Formula 1 or Formula 2), based on the weight of the composition. In a further embodiment, the composition comprises from 30 to 90 weight percent of the at least one compound, based on the weight of the composition. In a further embodiment, the composition comprises from 50 to 90 weight percent of the at least one compound, based on the weight of the composition.

In one embodiment, the composition further comprises an organometal compound, and further a metal quinolate. In a further embodiment, the metal quinolate is a lithium quinolate with or without substituents.

In one embodiment, the organometal compound comprises lithium. In a further embodiment, the organometal is lithium quinolate with or without substituents.

In one embodiment, the weight ratio of the at least one compound (Formula 1 or Formula 2) to the organometal compound is from 9/1 to 1/1, further from 7/1 to 1/1, further from 4/1 to 1/1, further from 3/2 to 1/1.

In one embodiment, the composition comprises from 10 to 90 weight percent of the metal quinolate, based on the weight of the composition. In a further embodiment, the composition comprises from 10 to 70 weight percent of the metal quinolate, based on the weight of the composition. In a further embodiment, the composition comprises from 10 to 50 weight percent of the metal quinolate, based on the weight of the composition. In a further embodiment, the composition comprises from 20 to 50 weight percent of the metal quinolate, based on the weight of the composition.

In one embodiment, the composition comprises from 10 to 90 weight percent of the lithium quinolate with or without substituents, based on the weight of the composition. In a further embodiment, the composition comprises 10 to 70 weight percent of the lithium quinolate, based on the weight of the composition. In a further embodiment, the composition comprises from 10 to 50 weight percent of the lithium quinolate, based on the weight of the composition. In a further embodiment, the composition comprises from 20 to 50 weight percent of the lithium quinolate, based on the weight of the composition.

The invention also provides a film comprising at least one Layer A formed from an inventive composition.

In one embodiment, the film further comprises another Layer B formed from a Composition B comprising at least one HTL (Hole Transport Layer) compound. In a further embodiment, Layer A is not adjacent to Layer B.

In one embodiment, the HIL (Hole Injection Layer) compound comprises an aromatic amine. In a further embodiment, the HIL compound is an aromatic diamine.

In one embodiment, Composition B comprises at least two HIL compounds. In a further embodiment, each HIL compound is independently a compound comprising an aromatic amine. In a further embodiment, each HIL compound is independently an aromatic diamine.

In one embodiment, Composition B does not contain a cyano compound (a compound comprising at least one cyano group).

In one embodiment, the inventive film comprises at least two aromatic amines, and further at least two aromatic diamines.

In one embodiment, each film layer is formed by a vacuum, thermal evaporation process.

An inventive compound may comprise a combination of two or more embodiments described herein.

An inventive composition may comprise a combination of two or more embodiments described herein.

Composition B may comprise a combination of two or more embodiments described herein.

Layer A may comprise a combination of two or more embodiments described herein.

Layer B may comprise a combination of two or more embodiments described herein.

An inventive film may comprise a combination of two or more embodiments described herein.

The invention also provides an article comprising at least one component formed from an inventive composition.

In one embodiment, the article is an electroluminescent device.

The invention also provides an article comprising at least one component formed from an inventive film.

In one embodiment, the article is an electroluminescent device.

An inventive article may comprise a combination of two or more embodiments described herein.

The inventive compounds may be used as charge transporting layers and as other layers in electronic devices, such as OLED devices. For example, the inventive compounds may be used as charge blocking layers and charge generation layers.

### Composition B

Composition B comprises at least one “HTL compound.” An HTL (Hole Transport Layer) compound is a material which transports holes. Typically the HTL has high hole mobility, and has the appropriate ionization potential to provide a small energy barrier for the following: hole injection from the anode or hole injection layer, and hole injection into the emitting layer; each leading to reduced operating voltage in the device. The HTL is also used to help block passage of electrons transported by the emitting layer. Small electron affinity is typically required to block electrons. The HTL should desirably have larger triplets to block exciton migration from an adjacent emitting layer. Examples of HTL compounds include, but are not limited to, di(p-tolyl)aminophenyl]-cyclohexane (TPAC), N,N-diphenyl-N,N-bis(3-methylphenyl)-1,1-biphenyl-4,4-diamine (TPD), and N,N′-diphenyl-N,N′-bis(1-naphthyl)-(1,1′-biphenyl)-4,4′-diamine (NPB).

In one embodiment, the HTL compound is selected from the following: 4,4′,4″-tris(N,N-(2-naphthyl)-phenylamino)triphenylamine (2-TNATA); N1,N1′-([1,1′-biphenyl]-4,4′-diyl)bis(N1-(naphthalen-1-yl)-N4,N4-diphenylbenzene-1,4-diamine); 4,4′,4″-Tris[phenyl(m-tolyl)amino]triphenylamine (m-MTDATA); or N4,N4′-Bis[4-[bis(3-methylphenyl)amino]phenyl]-N4,N4′-diphenyl-[1,1′-biphenyl]-4,4′-diamine (DNTPD).

In one embodiment, the HTL compound is selected from the following: di(p-tolyl)amino-phenyl]cyclohexane (TPAC); N,N-diphenyl-N,N-bis(3-methylphenyl)-1,1-biphenyl-4,4-diamine (TPD); or N,N′-diphenyl-N,N′-bis(1-naphthyl)-(1,1′-biphenyl)-4,4′-diamine (NPB). In a further embodiment, the HTL compound is selected from the following: N,N-diphenyl-N,N-bis(3-methylphenyl)-1,1-biphenyl-4,4-diamine(TPD); or N,N′-diphenyl-N,N′-bis(1-naphthyl)-(1,1′-biphenyl)-4,4′-diamine(NPB). In a further embodiment, the HTL compound is N,N′-diphenyl-N,N′-bis(1-naphthyl)-(1,1′-biphenyl)-4,4′-diamine (NPB).

In one embodiment, Composition B comprises 100 weight percent of the HTL compound, based on the weight of Composition B.

Composition B may comprise a combination of two or more embodiments described herein.

Layer B formed from Composition B may comprise a combination of two or more embodiment described herein.

### DEFINITIONS

The term “aryl,” described herein, represents an organic radical derived from aromatic hydrocarbon by deleting one hydrogen atom therefrom. An aryl group may be a monocyclic and/or fused ring system, each ring of which suitably contains from 4 to 7, preferably from 5 or 6 atoms. Structures wherein two or more aryl groups are combined through single bond(s) are also included. Specific examples include, but are not limited to, phenyl, naphthyl, biphenyl, anthryl, indenyl, fluorenyl, benzofluorenyl, phenanthryl, triphenylenyl, pyrenyl, perylenyl, chrysenyl, naphtacenyl, fluoranthenyl and the like, but are not restricted thereto. The naphthyl may be 1-naphthyl or 2-naphthyl, the anthryl may be 1-anthryl, 2-anthryl or 9-anthryl, and the fluorenyl may be any one of 1-fluorenyl, 2-fluorenyl, 3-fluorenyl, 4-fluorenyl and 9-fluorenyl.

The term “heteroaryl” described herein refers to an aryl group containing at least one heteroatom, for example, B, N, O, S, P(═O), Si and P, for the aromatic cyclic backbone atoms, and carbon atom(s) for remaining aromatic cyclic backbone atoms. The heteroaryl may be a 5- or 6-membered monocyclic heteroaryl or a polycyclic heteroaryl, which is fused with one or more benzene ring(s), and may be partially saturated. The structures having one or more heteroaryl group(s) bonded through a single bond are also included. The heteroaryl groups may include divalent aryl groups of which the heteroatoms are oxidized or quarternized to form N-oxides, quaternary salts, or the like. Specific examples include, but are not limited to, monocyclic heteroaryl groups, such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl; polycyclic heteroaryl groups, such as benzofuranyl, fluoreno[4,3-b]benzofuranyl, benzothiophenyl, fluoreno[4,3-b]benzothiophenyl, isobenzofuranyl, benzimidazolyl, benzothiazolyl, benzisothiazolyl, benzisoxazolyl, benzoxazolyl, isoindolyl, indolyl, indazolyl, benzothia-diazolyl, quinolyl, isoquinolyl, cinnolinyl, quinazolinyl, quinoxalinyl, carbazolyl, phenanthridinyl and benzodioxolyl; and corresponding N-oxides (for example, pyridyl N-oxide, quinolyl N-oxide) and quaternary salts thereof.

Substituents include, but are not limited to, the following: deuterium, halogen, (C1-C30)alkyl with or without halogen substituent(s), (C6-C30)aryl, (C3-C30)heteroaryl with or without (C6-C30)aryl substituent(s), a 5- to 7-membered heterocycloalkyl containing one or more heteroatom(s) selected from, for example, B, N, O, S, P(═O), Si and P, a 5- to 7-membered heterocycloalkyl fused with one or more aromatic ring(s), (C3-C30)cycloalkyl, (C5-C30)cycloalkyl fused with one or more aromatic ring(s), tri(C1-C30)alkylsilyl, di(C1-C30)alkyl(C6-C30)arylsilyl, tri(C6-C30)arylsilyl, adamantyl, (C7-C30)bicycloalkyl, (C2-C30)alkenyl, (C2-C30)alkynyl, cyano, carbazolyl, NR₂iR₂₂, BR₂₃R₂₄, PR₂₅R₂₆, P(═O)R₂₇R₂₈ [wherein R₂i through R₂₈ independently represent (C1-C30)alkyl, (C6-C30)aryl or (C3-C30)heteroaryl], (C6-C30)ar(C1-C30)alkyl, (C1-C30)alkyl(C6-C30)aryl, (C1-C30)alkyloxy, (C1-C30)alkylthio, (C6-C30)aryloxy, (C6-C30)arylthio, (C1-C30)alkoxycarbonyl, (C1-C30)alkylcarbonyl, (C6-C30)arylcarbonyl, (C6-C30)aryloxycarbonyl, (C1-C30)alkoxycarbonyloxy, (C1-C30)alkylcarbonyloxy, (C6-C30)arylcarbonyloxy, (C6-C30)aryloxycarbonyloxy, carboxyl, nitro and hydroxyl; or that the adjacent substituents are linked together to form a ring. For example, a substituent may form a ring structure with one or more atoms on the backbone molecule comprising said substituent.

### EXPERIMENTAL

### Reagents and Test Methods

All solvents and reagents were obtained from commercial vendors, including Sigma-Aldrich, TCI, and Alfa Aesar, and were used in the highest available purities, and/or when necessary, recrystallized before use. Dry solvents were obtained from in-house purification/dispensing system (hexane, toluene, and tetrahydrofuran), or purchased from Sigma-Aldrich. All experiments involving “water sensitive compounds” were conducted in “oven dried” glassware, under nitrogen atmosphere, or in a glovebox. Reactions were monitored by analytical, thin-layer chromatography (TLC) on precoated aluminum plates (VWR 60 F254), and visualized by UV light and/or potassium permanganate staining. Flash chromatography was performed on an ISCO COMBIFLASH system with GRACERESOLV cartridges. GC-mass spectrometry (GC-MS) was performed on a HP 6890 series GC system with a “12 m×0.2 mm×0.55 μM” DB-MS column (coiled).

¹H-NMR-spectra (500 MHz or 400 MHz) were obtained on a Varian VNMRS-500 or VNMRS-400 spectrometer, at 30° C., unless otherwise noted. The chemical shifts were referenced to TMS (δ=0.00) in CDCl₃.

¹³C-NMR spectra (125 MHz or 100 MHz) were obtained on a Varian VNMRS-500 or VNRMS-400 spectrometer, and referenced to TMS (δ=0.00) in CDCl₃.

Routine LC/MS studies were carried out as follows. Five microliter aliquots of the sample, as “3 mg/ml solution in THF,” were injected on an AGILENT 1200SL binary gradient, liquid chromatography, coupled to an AGILENT 6520 QTof, quadruple-time of flight MS system, via a dual spray electrospray (ESI) interface, operating in the PI mode. The following analysis conditions were used: column: 150×4.6 mm ID, 3.5 μm ZORBAX SB-C8; column temperature: 40° C.; mobile phase: 75/25 A/B to 15/85 A/B at 40 minutes; solvent A=0.1 v % formic acid in water; solvent B=THF; flow 1.0 mL/min; UV detection: diode array 210 to 600 nm (extracted wavelength 250,280 nm); ESI conditions: gas temperature 365° C.; gas flow—8 ml/min; capillary—3.5 kV; nebulizer—40PSI; fragmentor −145V.

DSC was done using a 2000 instrument at a scan rate of 10° C./min, and in a nitrogen atmosphere for all cycles. The sample (about 7-10 mg) was scanned from room temperature to 300° C., cooled to −60° C., and reheated to 300° C. The glass transition temperature (T_{g}) was measured on the second heating scan. Data analysis was performed using TA Universal Analysis software. The T_{g} was calculated using the “mid-point of inflection” methodology.

### Modeling

All computations utilized the Gaussian09 program¹. The calculations were performed with the hybrid density functional theory (DFT) method, B3LYP,² and the 6-31G* (5d) basis set.³ The singlet state calculations used the closed shell approximation, and the triplet state calculations used the open shell approximation. All values are quoted in electronvolts (eV). The HOMO and LUMO values were determined from the orbital energies of the optimized geometry of the singlet ground state. The triplet energies were determined as the difference between the total energy of the optimized triplet state and the optimized singlet state.
1. Gaussian 09, Revision A.02, Frisch, M. J.; Trucks, G. W.; Schlegel, H. B.; Scuseria, G. E.; Robb, M. A.; Cheeseman, J. R.; Scalmani, G.; Barone, V.; Mennucci, B.; Petersson, G. A.; Nakatsuji, H.; Caricato, M.; Li, X.; Hratchian, H. P.; Izmaylov, A. F.; Bloino, J.; Zheng, G.; Sonnenberg, J. L.; Hada, M.; Ehara, M.; Toyota, K.; Fukuda, R.; Hasegawa, J.; Ishida, M.; Nakajima, T.; Honda, Y.; Kitao, O.; Nakai, N.; Vreven, T.; Montgomery, Jr., J. A.; Peralta, J. E.; Ogliaro, F.; Bearpark, M.; Heyd, J. J.; Brothers, E.; Kudin, K. N.; Staroverov, V. N.; Kobayashi, R.; Normand, J.; Raghavachari, K.; Rendell, A.; Burant, J. C.; Iyengar, S. S.; Tomasi, J.; Cossi, M.; Rega, N.; Millam, J. M.; Klene, M.; Knox, J. E.; Cross, J. B.; Bakken, V.; Adamo, C.; Jaramillo, J.; Gomperts, R.; Stratmann, R. E.; Yazyev, O.; Austin, A. J.; Cammi, R.; Pomelli, C.; Ochterski, J. W.; Martin, R. L.; Morokuma, K.; Zakrzewski, V. G.; Voth, G. A.; Salvador, P.; Dannenberg, J. J.; Dapprich, S.; Daniels, A. D.; Farkas, O.; Foresman, J. B.; Ortiz, J. V.; Cioslowski, J.; Fox, D. J., Gaussian, Inc., Wallingford Conn., 2009.
2. (a) Becke, A. D. J. Chem. Phys. 1993, 98, 5648. (b) Lee, C.; Yang, W.; Parr, R. G. Phys. Rev B 1988, 37, 785. (c) Miehlich, B.; Savin, A.; Stoll, H.; Preuss, H. Chem. Phys. Lett. 1989, 157, 200.
3. (a) Ditchfield, R.; Hehre, W. J.; Pople, J. A. J. Chem. Phys. 1971, 54, 724. (b) Hehre, W. J.; Ditchfield, R.; Pople, J. A. J. Chem. Phys. 1972, 56, 2257. (c) Gordon, M. S. Chem. Phys. Lett. 1980, 76, 163.

Many of the inventive compounds can be synthesized, in part, by using the following: standard transformations of cyanuric chloride and aryl Grignards; metal-mediated coupling of aryl halides with organoboron reactants; cyclotrimerization of carbonitriles, or a combination thereof. The inventive compounds can be used as charge transporting layers and other layers in an OLED device, for example, as charge blocking layers and charge generation layers.

### I. Preparation of Intermediate Compound Ia

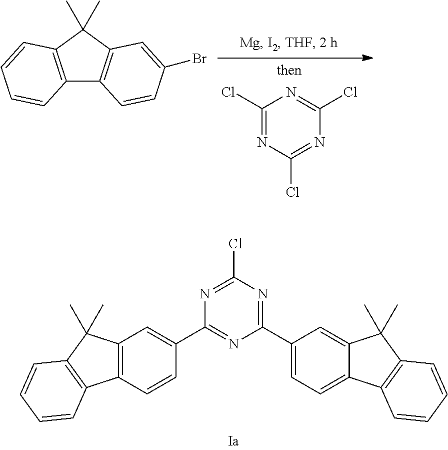 [SMILES: CC1(C)c2ccccc2-c2ccc(Br)cc21 ; Clc1nc(Cl)nc(Cl)n1 ; CC1(C)c2ccccc2-c2ccc(-c3nc(Cl)nc(-c4ccc5c(c4)C(C)(C)c4ccccc4-5)n3)cc21]

In a glove box, 2-bromo-9,9-dimethylfluorene (11 g, 40 mmol) was dissolved in 100 mL of dry THF. Magnesium turnings (1.9 g, 80 mmol) and a crystal of iodine were added into the solution, and stirred at 55° C. After 1 h, GC-MS analysis of an aliquot of the reaction mixture (quenched with water and extracted with EtOAc) indicated complete conversion to the expected Grignard. This solution was added to a solution of 1,3,5-trichlorotriazine (3.68 g, 20 mmol) in THF (10 mL) (exotherm observed). Aliquots of the reaction were analyzed by LC-MS for 1 hour, after which, more THF (40 mL) was added to the mixture, and the reaction was stirred at that temperature overnight. LC-MS analysis of an aliquot of the product, after overnight stirring, indicated >95% conversion of starting material to the desired product. The solvent was removed, under reduced pressure, and the product was dissolved in hot chloroform, washed with water, the solvent removed under reduced pressure, and the product purified using column chromatography (10-40% chloroform/hexanes gradient) to give the desired compound Ia (9 g, 90% isolated yield).

### II. Preparation of Intermediate Compound Ib

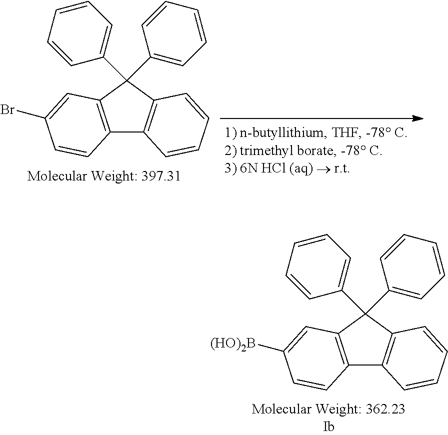 [SMILES: Brc1ccc2c(c1)C(c1ccccc1)(c1ccccc1)c1ccccc1-2 ; OB(O)c1ccc2c(c1)C(c1ccccc1)(c1ccccc1)c1ccccc1-2]

The compound 2-bromo-9,9-diphenyl-9H-fluorene (3.00 g, 7.55 mmol) was dissolved in anhydrous THF (150 mL), and n-butyl lithium (6.14 mL, 1.6 M in hexanes, 1.3 eq.) was added to the solution at −78° C. The mixture solution was stirred at −78° C. for 2 h. To the solution, trimethyl borate (1.1 mL, 1.3 eq.) was added at −78° C. After stirring the solution for 1 h, 5 mL of 6 N HCl solution was added to the solution, and the resulting biphasic solution was stirred rapidly for 2 h at room temperature. After the reaction, the organic layer was extracted with methylene chloride, and washed with brine, dried over MgSO₄, and the solvent removed under vacuum. The crude product was purified by silica chromatogram with methylene chloride, and the mixture of methylene chloride and methanol (9:1). After purification, 2 g of compound Ib white powder was obtained (73.3% yield).

### III. Synthesis of Inventive Compound I

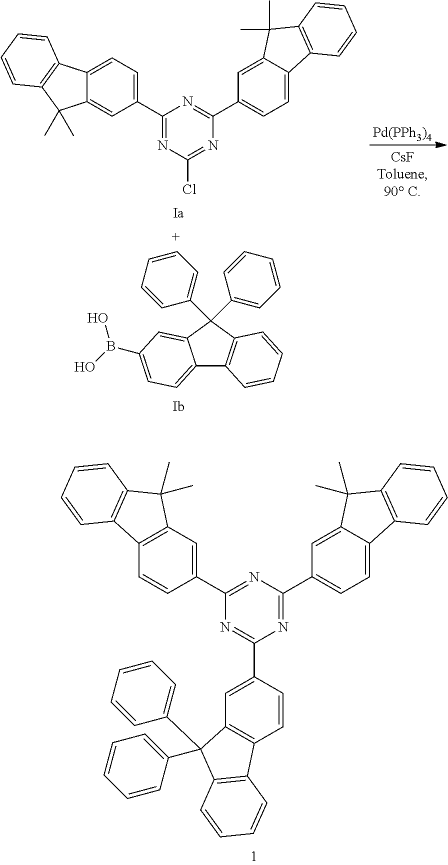 [SMILES: CC1(C)c2ccccc2-c2ccc(-c3nc(Cl)nc(-c4ccc5c(c4)C(C)(C)c4ccccc4-5)n3)cc21 ; OB(O)c1ccc2c(c1)C(C1=C[C+]=CC=C1)(c1ccccc1)c1ccccc1-2 ; CC1(C)c2ccccc2-c2ccc(-c3nc(-c4ccc5c(c4)C(C)(C)c4ccccc4-5)nc(-c4ccc5c(c4)C(c4ccccc4)(c4ccccc4)c4ccccc4-5)n3)cc21 ; "Pd(PPh3)4"]

In a glovebox, a magnetically stirred mixture of 3,5-bis(9,9-dimethylfluorenyl)-1-chlorotriazine 1a (2.12 g, 4.25 mmol), (9,9-diphenyl-9H-fluoren-2-yl)boronic acid 1b (2.00 g, 5.52 mmol, 1.3 equivalents), and CsF (1.94 g, 12.74 mmol) in 100 mL of dry toluene, was charged with Pd(PPh₃)₄ (250 mg, 0.21 mmol, 4.94 mol %). The reaction mixture was heated at 90° C., and aliquots periodically analyzed by LC-MS to check reaction progress. After 12 h, LC-MS analysis showed complete conversion of starting material to expected product. The reaction mixture was cooled down, diluted with methylene chloride (300 mL), the salts washed off with water (200 mL), and the aqueous layer extracted with methylene chloride. After solvent removal under vacuum, the crude product was purified by a silica column separation with 30% methylene chloride in hexanes. After column separation, the product was dissolved in THF, and the mixture solution was mixed with 10% NaOH aqueous solution. The mixture solution was refluxed for 1 h. After cooling, methylene chloride was added to the solution, and washed with water. After removal of the organic solvent, 2 g of pure inventive compound 1 was obtained (63.25%; 99.40% pure before sublimation).

¹H NMR (400 MHz, CHLOROFORM-d) δ 1.65 (s, 12H) 7.25-7.46 (m, 16H), 7.49-7.55 (m, 3H) 7.81-7.88 (m, 2H) 7.88-7.95 (m, 3H) 8.01 (dd, J=1.00, 1.00 Hz, 1H), 8.78-8.82 (m, 4H) 8.84-8.87 (m, 2H). ¹³C NMR (101 MHz, CHLOROFORM-d) δ 27.19, 47.03, 65.66, 120.07, 120.81, 122.80, 123.08, 126.44, 126. 86, 127.18, 127.71, 128.21, 128.37, 128.45, 128.69, 128.84, 135.29, 136.07, 138.46, 139.39, 143.59, 144.45, 145.71, 151.50, 152.27, 153.85, 154.73, 171.50, 171.64.

MW=782 g/mole; Tg=168° C.; HOMO=−5.79 eV; LUMO=−1.85 eV; Triplet Energy=2.59 eV.

### IV. Preparation of Intermediate Compound Ic

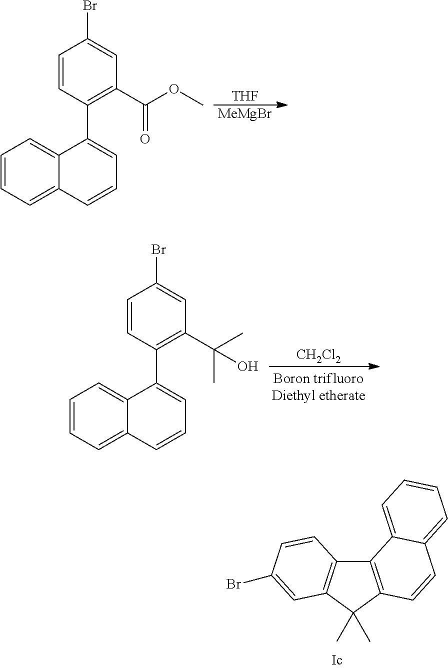

A 3 neck round bottom flask was charged with the starting benzoate (1.00 grams, 2.93 mmol), a stir bar and anhydrous THF (˜20 mL). This flask was taken out of the glove box, and equipped with an addition funnel, condenser, nitrogen inlet and a septa. The round bottom flask was placed in an ice bath and allowed to stir. The addition funnel then had 2.93 mL of MeMgBr (8.79 mmol) added thereto. The MeMgBr solution was then added to the benzoate solution, drop wise, over a one hour period. Then the reaction vessel was allowed to warm to room temp, and left to stir overnight. The reaction was tested by taking an aliquot, quenched with water, and taken up in ethyl acetate. Once completed, the crude material was loaded onto a silica column with a 5% ethyl acetate isocratic run. The first intermediate product was confirmed by GC/MS and NMR.

To a solution of naphthyl phenyl propanol (6.27 g, 18.37 mmol), in dichloromethane (˜50 mL), was added trifluoroboron etherate (2.27 mL, 18.37 mmol) slowly, via syringe, and the solution was stirred overnight at room temperature. The reaction was quenched with methanol, and evaporated under reduced pressure. Quenched with 1 mL of methanol. The reaction mixture was run through a small plug and reduced down. The crude material was confirmed by GC/MS. It was determined that the second peak on the GC/MS trace was the six member ring closure variant. This was removed by re-crystallization from hexanes. The product was obtained intermediate compound Ic (96% yield).

### V. Preparation of Intermediate Compound Id

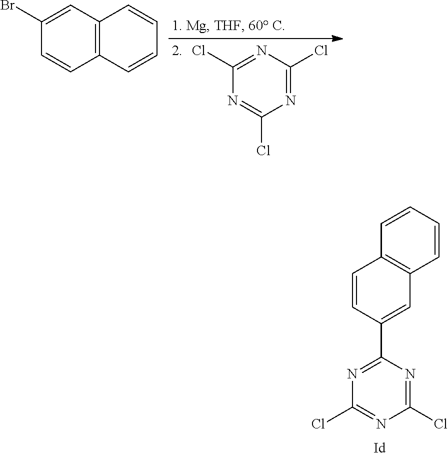 [SMILES: Brc1ccc2ccccc2c1 ; Clc1nc(Cl)nc(Cl)n1 ; Clc1nc(Cl)nc(-c2ccc3ccccc3c2)n1]

In a glovebox, a vial was charged with 2-bromonaphthalene (6.2 g, 30 mmol), 60 mL of dry THF, magnesium turnings (0.875 g, 36 mmol), and a small crystal of iodine. The reaction was stirred at 50° C. The iodide color disappeared after a few minutes (solution becomes clear), and then slowly became greenish-yellow in color. GC-MS analysis of an aliquot of the reaction mixture (quenched with D₂O and extracted with EtOAc) was done periodically to check the reaction progress. Upon complete conversion, the Grignard reaction was slowly transferred, by Pasteur pipette, to a solution of trichlorotriazine (11 g, 60 mmol) in THF (60 mL; caution: exothermic reaction). The solution immediately turned dark brown, and slowly changed to dark purple in color; and the reaction mixture was heated at 50° C., with periodic LC-MS monitoring. The solvent was dried, and the solid was first precipitated with methanol/ethyl acetate to remove some of the trichlorotriazine, and then redissolved in chloroform, and then dry silica gel was added to make a slurry, which was then dried to a free-flowing powder. The powder was packed into a cartridge, and loaded on to an ISCO purification system, and the desired product was eluted with chloroform-hexane (30-50% CHCl3/hexanes) solvent system. The fractions, having the desired compound, were combined, and the solvent removed by rotary evaporation, to give a white powder (6.5 g, 78% yield, ˜98% pure) of the desired compound Id, as determined by LC-MS and ¹H-NMR.

### VI. Synthesis of Intermediate Ie

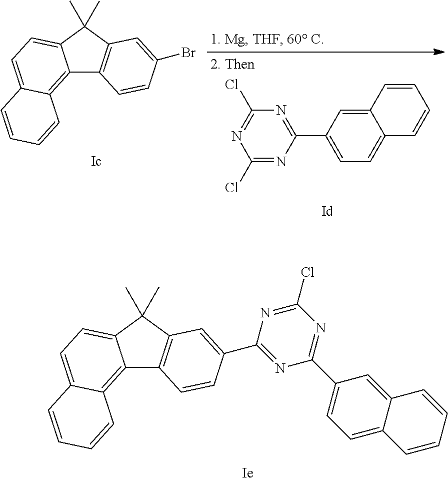 [SMILES: CC1(C)c2cc(Br)ccc2-c2c1ccc1ccccc21 ; Clc1nc(Cl)nc(-c2ccc3ccccc3c2)n1 ; CC1(C)c2cc(-c3nc(Cl)nc(-c4ccc5ccccc5c4)n3)ccc2-c2c1ccc1ccccc21]

A glass jar was charged with 9-bromo-7,7-dimethyl-7H-benzo[c]fluorine 4 (3.23 g, 10 mmol), 30 mL of dry THF, and magnesium turnings (0.48 g, 20 mmol), and the reaction mixture was stirred at 50° C. under nitrogen. GC-MS analysis of an aliquot of the reaction mixture (quenched with D₂O, and extracted with EtOAc) was done periodically to check the reaction progress. Upon complete conversion, this Grignard solution was slowly added to a solution of 2,4-dichloro-6-(naphthalen-2-yl)-1,3,5-triazine (2.76 g, 10 mmol), in dry THF (30 mL), at room temperature. The reaction mixture was stirred, at room temperature, with periodic analysis by LC-MS, until complete conversion. The solvent was removed by rotatory evaporation, and the solid was redissolved in chloroform, dried loaded with silica gel, and purified by silica gel chromatography with chloroform/hexanes gradient (20-40%), to give the desired intermediate compound Ie (3.1 g, 64% yield).

### VII. Preparation of Inventive Compound 2

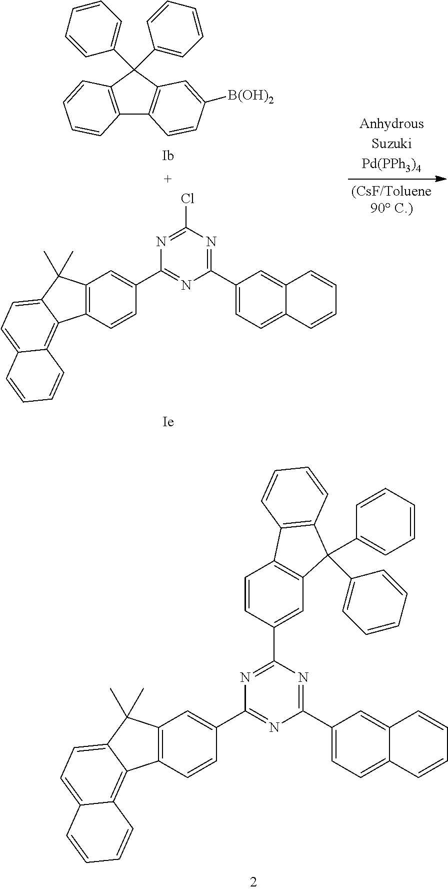 [SMILES: OB(O)c1ccc2c(c1)C(c1ccccc1)(c1ccccc1)c1ccccc1-2 ; CC1(C)c2cc(-c3nc(Cl)nc(-c4ccc5ccccc5c4)n3)ccc2-c2c1ccc1ccccc21 ; CC1(C)c2cc(-c3nc(-c4ccc5c(c4)C(c4ccccc4)(c4ccccc4)c4ccccc4-5)nc(-c4ccc5ccccc5c4)n3)ccc2-c2c1ccc1ccccc21]

In a glove box, a glass jar was charged with a mixture of 2-chloro-4-(9,9-dimethyl-9H-fluoren-2-yl)-6-(naphthalen-2-yl)-1,3,5-triazine 5 (2.17 g, 5 mmol), 9,9-diphenyl-9H-fluoren-2-ylboronic acid 9 (2 g, 5.5 mmol, 1.1 equivalents), and powdered CsF (Sigma Aldrich) (1.52 g, 10 mmol, 2 equivalents). Dry toluene (70 mL) was added, followed with Pd(PPh₃)₄ (Sigma Aldrich) (289 mg, 0.25 mmol, 5 mol %). The reaction mixture was heated to 90° C. for 2 h, after which, analysis of an aliquot of the reaction by LC-MS, indicated complete conversion to the desired product.

The solvent was removed by rotatory evaporation, and the solid was redissolved in chloroform, dried loaded with silica gel and purified by silica gel chromatography, with chloroform/hexanes gradient (10-30%), to give the desired inventive compound 2 (3.3 g, 86% yield). 1H NMR (500 MHz, Chloroform-d) δ 9.32 (dd, J=1.6, 0.8 Hz, 1H), 8.94 (d, J=1.4 Hz, 1H), 8.91-8.85 (m, 3H), 8.82 (ddd, J=8.3, 6.7, 1.4 Hz, 2H), 8.52-8.45 (m, 1H), 8.14-8.08 (m, 1H), 8.06-7.88 (m, 6H), 7.73-7.65 (m, 2H), 7.64-7.52 (m, 4H), 7.51-7.42 (m, 5H), 7.42-7.29 (m, 7H), 1.73 (s, 6H). 13C NMR (126 MHz, Chloroform-d) δ 171.58, 171.50, 171.39, 154.86, 154.04, 152.28, 151.53, 145.80, 144.64, 144.52, 139.42, 136.05, 135.66, 134.11, 133.77, 133.66, 133.10, 132.71, 130.00, 129.81, 129.57, 129.50, 129.37, 129.32, 128.93, 128.73, 128.64, 128.52, 128.45, 128.31, 128.29, 127.87, 127.79, 127.75, 127.04, 127.01, 126.94, 126.91, 126.49, 126.42, 125.28, 125.21, 123.88, 123.04, 122.63, 121.07, 120.87, 120.30, 98.80, 65.73, 46.87, 26.72.

MW=765 g/mole; Tg=175° C.; HOMO=−5.47 eV; LUMO=−1.93 eV; Triplet Energy=2.2 eV.

### VIII. Alternative Preparation of Inventive Compound 2: Synthesis of Intermediate If

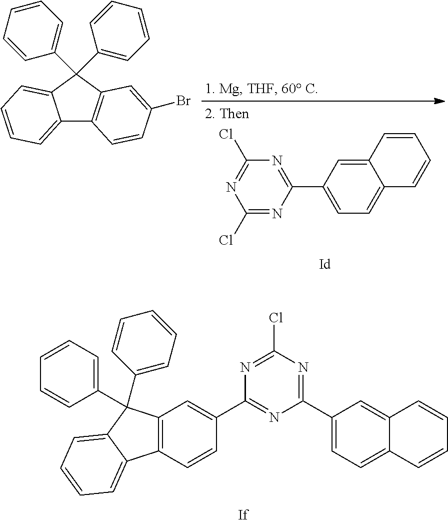 [SMILES: Brc1ccc2c(c1)C(c1ccccc1)(c1ccccc1)c1ccccc1-2 ; Clc1nc(Cl)nc(-c2ccc3ccccc3c2)n1 ; Clc1nc(-c2ccc3c(c2)C(c2ccccc2)(c2ccccc2)c2ccccc2-3)nc(-c2ccc3ccccc3c2)n1]

A dry glass jar was charged with 2-bromo-9,9-diphenyl-9H-fluorene (3.23 g, 10 mmol), 40 mL of dry THF, and magnesium turnings (0.36 g, 15 mmol), and the reaction mixture was stirred at 50° C. under nitrogen. GC-MS analysis of an aliquot of the reaction mixture (quenched with D₂O and extracted with EtOAc) was done periodically to check the reaction progress. Upon complete conversion, this Grignard solution was slowly added to a solution of 2,4-dichloro-6-(naphthalen-2-yl)-1,3,5-triazine Id (2.76 g, 10 mmol) in dry THF (20 mL), at room temperature. The reaction mixture was stirred at room temperature, with periodic analysis by LC-MS, until complete conversion. The solvent was removed by rotatory evaporation, and the solid was redissolved in chloroform, dried loaded with silica gel, and purified by silica gel chromatography with chloroform/hexanes gradient (20-40%), to give the desired intermediate compound If (3.5 g, 63% yield).

### IX. Alternative Preparation of Inventive Compound 2: Synthesis of Intermediate Ig

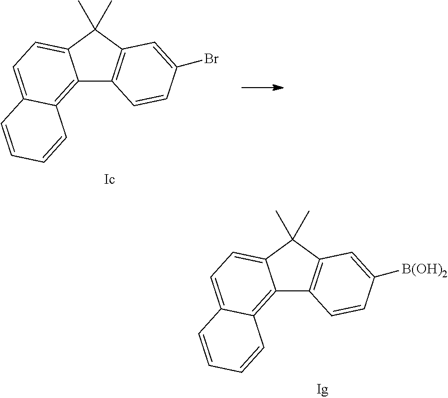 [SMILES: CC1(C)c2cc(Br)ccc2-c2c1ccc1ccccc21 ; CC1(C)c2cc(B(O)O)ccc2-c2c1ccc1ccccc21]

A 3 neck 100 L RB was loaded with 5.0 grams (15 mmol) of the 5-bromo-2-benzofluorene Ic. The flask was then outfitted with a stir, thermal well, and a condenser with a nitrogen inlet. The reaction flask was purged with nitrogen for one hour. Then, 50 mL of anhydrous THF was added via syringe. The flask was then cooled to 5-10° C. with an ice bath. Then, 10.15 mL of 1.6 M BuLi (16 mmol, 1.05 equivalents) in hexane was added dropwise, over 60 minutes, with a syringe pump. After the addition was complete, the reaction was allowed to stir for one hour, and then neat trimethyl borate (19.3 mmol) was added over a 15 minute period. The reaction was allowed to stir for approximately one hour, at ambient temperatures, and then approximately 2.2 mL of 45% KOH was added dropwise over five minutes. The reaction was allowed to stand overnight. The reaction mixture was extracted with ethyl acetate. Approximately 4 mL of concentrated HCl (48 mmol) was added dropwise, over 10 minutes, to the stirring reaction, followed by 50 mL of ethyl acetate. The organic phase was separated, dried with magnesium sulfate, and then the solvent was removed to yield 4.8 grams of a white powder. The product appeared to be a mixture of the desired product, byproduct, and residual ethyl acetate. The product was utilized without further purification.

### X. Alternative Preparation of Inventive Compound 2

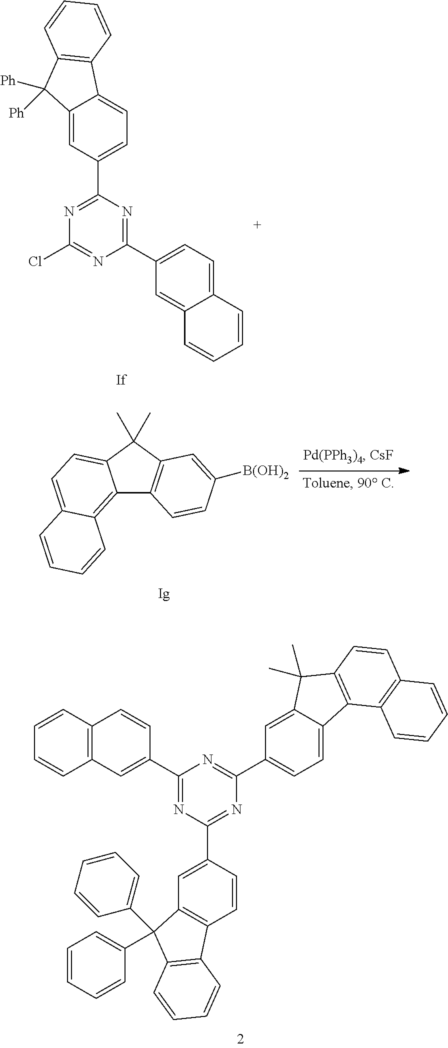 [SMILES: Clc1nc(-c2ccc3c(c2)C(c2ccccc2)(c2ccccc2)c2ccccc2-3)nc(-c2ccc3ccccc3c2)n1 ; CC1(C)c2cc(B(O)O)ccc2-c2c1ccc1ccccc21 ; CC1(C)c2cc(-c3nc(-c4ccc5c(c4)C(c4ccccc4)(c4ccccc4)c4ccccc4-5)nc(-c4ccc5ccccc5c4)n3)ccc2-c2c1ccc1ccccc21]

In a glove box, a glass jar was charged with a mixture of 2-chloro-4-(9,9-diphenyl-9H-fluoren-2-yl)-6-(naphthalen-2-yl)-1,3,5-triazine If (2 g, 1.95 mmol), 7,7-dimethyl-7H-benzo[c]fluoren-9-ylboronic acid Ig (1.1 g, 3.85 mmol, 1.1 equivalents), and powdered CsF (Chemtal) (1.1 g, 7 mmol, 2 equivalents). Dry toluene (40 mL) was added, followed with Pd(PPh3)4 (Sigma Aldrich) (202 g, 0.175 mmol, 5 mol %). The reaction mixture was heated to 90° C., and aliquots were periodically analyzed by LC-MS to check reaction progress. The solvent was removed by rotatory evaporation, and the solid was redissolved in chloroform, dried loaded with silica gel, and purified by silica gel chromatography, with chloroform/hexanes gradient (10-30%), to give the desired inventive compound 2 (1.1 g, 74% yield, 99% yield by LC-MS).

1H NMR (500 MHz, Chloroform-d) δ 9.32 (dd, J=1.6, 0.8 Hz, 1H), 8.94 (d, J=1.4 Hz, 1H), 8.91-8.85 (m, 3H), 8.82 (ddd, J=8.3, 6.7, 1.4 Hz, 2H), 8.52-8.45 (m, 1H), 8.14-8.08 (m, 1H), 8.06-7.88 (m, 6H), 7.73-7.65 (m, 2H), 7.64-7.52 (m, 4H), 7.51-7.42 (m, 5H), 7.42-7.29 (m, 7H), 1.73 (s, 6H). 13C NMR (126 MHz, Chloroform-d) δ 171.58, 171.50, 171.39, 154.86, 154.04, 152.28, 151.53, 145.80, 144.64, 144.52, 139.42, 136.05, 135.66, 134.11, 133.77, 133.66, 133.10, 132.71, 130.00, 129.81, 129.57, 129.50, 129.37, 129.32, 128.93, 128.73, 128.64, 128.52, 128.45, 128.31, 128.29, 127.87, 127.79, 127.75, 127.04, 127.01, 126.94, 126.91, 126.49, 126.42, 125.28, 125.21, 123.88, 123.04, 122.63, 121.07, 120.87, 120.30, 98.80, 65.73, 46.87, 26.72.

### XI. Preparation of Inventive Compound 3

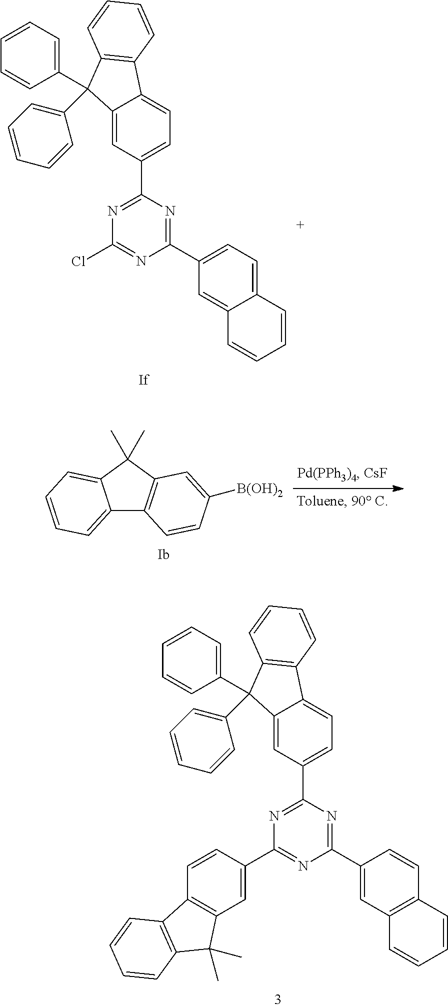 [SMILES: Clc1nc(-c2ccc3c(c2)C(c2ccccc2)(c2ccccc2)c2ccccc2-3)nc(-c2ccc3ccccc3c2)n1 ; CC1(C)c2ccccc2-c2ccc(B(O)O)cc21 ; CC1(C)c2ccccc2-c2ccc(-c3nc(-c4ccc5c(c4)C(c4ccccc4)(c4ccccc4)c4ccccc4-5)nc(-c4ccc5ccccc5c4)n3)cc21]

In a glove box, a glass jar was charged with a mixture of 2-chloro-4-(9,9-diphenyl-9H-fluoren-2-yl)-6-(naphthalen-2-yl)-1,3,5-triazine If (2.8 g, 5 mmol), 9,9-dimethyl-9H-fluoren-2-ylboronic acid Ib (1.3 g, 5.5 mmol, 1.1 equivalents), and powdered CsF (Chemtal) (1.52 g, 10 mmol, 2 equivalents). Dry toluene (60 mL) was added, followed with Pd(PPh3)4 (Sigma Aldrich) (0.288 g, 0.25 mmol, 5 mol %). The reaction mixture was heated to 90° C., and aliquots were periodically analyzed by LC-MS to check reaction progress. The solvent was removed by rotatory evaporation, and the solid was redissolved in chloroform, dried loaded with silica gel, and purified by silica gel chromatography with chloroform/hexanes gradient (10-30%), to give the desired inventive compound 3 (3 g, 86% yield).

¹H NMR (400 MHz, Chloroform-d) δ 9.30 (dd, J=1.5, 0.8 Hz, 1H), 8.94-8.84 (m, 2H), 8.84-8.75 (m, 3H), 8.14-8.05 (m, 1H), 8.06-7.96 (m, 2H), 7.96-7.87 (m, 3H), 7.88-7.78 (m, 1H), 7.66-7.54 (m, 2H), 7.54-7.48 (m, 2H), 7.48-7.35 (m, 7H), 7.35-7.20 (m, 6H), 1.65 (s, 6H). ¹³C NMR (101 MHz, Chloroform-d) δ 171.86, 171.70, 171.58, 154.88, 154.01, 152.39, 151.66, 145.86, 144.67, 143.79, 139.51, 138.58, 136.14, 135.79, 135.36, 133.88, 133.23, 130.13, 129.69, 129.04, 128.86, 128.64, 128.54, 128.44, 128.39, 128.37, 127.99, 127.94, 127.86, 127.32, 127.02, 126.60, 126.56, 125.31, 123.23, 122.94, 121.17, 120.97, 120.42, 120.23, 76.84, 65.82, 47.20, 27.33.

MW=716 g/mole; Tg=165° C.; HOMO=−5.81 eV; LUMO=−1.89 eV; Triplet Energy=2.50 eV.

### OLED Device Fabrication and Testing

All organic materials were purified by sublimation before deposition. OLEDs were fabricated onto an ITO coated glass substrate that served as the anode, and topped with an aluminum cathode. All organic layers were thermally deposited by chemical vapor deposition in a vacuum chamber with a base pressure of <10⁻⁷ torr. The deposition rates of organic layers were maintained at 0.1˜0.05 nm/s. The aluminum cathode was deposited at 0.5 nm/s. The active area of the OLED device was “3 mm×3 mm,” as defined by the shadow mask for cathode deposition.

Each cell, containing HIL, HTL, EML host, EML dopant, ETL, or EIL, was placed inside a vacuum chamber, until it reached 10⁻⁶ torr. To evaporate each material, a controlled current was applied to the cell, containing the material, to raise the temperature of the cell. An adequate temperature was applied to keep the evaporation rate of the materials constant throughout the evaporation process.

For the HIL layer, N1,N1′-([1,1′-biphenyl]-4,4′-diyl)bis(N1-(naphthalen-1-yl)-N4,N4-diphenylbenzene-1,4-diamine) was evaporated at a constant 1 A/s rate, until the thickness of the layer reached 600 Angstrom. Simultaneously, the N4,N4′-di(naphthalen-1-yl)-N4,N4′-diphenyl-[1,1′-biphenyl]-4,4′-diamine (NPB) layer was evaporated at a constant 1 A/s rate, until the thickness reached 200 Angstrom. For the EML layer, 9,10-di(naphthalen-2-yl)anthracene (ADN, host) and (E)-4,4′-(ethene-1,2-diyl)bis(N,N-diphenylaniline) (DPAVB, dopant) were co-evaporated, until the thickness reached 350 Angstrom. The deposition rate for host material was 0.98 A/s, and the deposition for the dopant material was 0.02 A/s, resulting in a 2% doping of the host material. For the ETL layer, the ETL compounds were co-evaporated with lithium quinolate(Liq), until the thickness reached 300 Angstrom. The evaporation rate for the ETL compounds and Liq was 0.5 A/s. Alq₃ (tris(8-hydroxyquinolinato)aluminum) was used as a reference material to compare with the inventive compounds. Alq3 was evaporated solely at 1 A/s rate, until 300 Angstrom. Finally, “20 Angstrom” of a thin electron injection layer (Liq) was evaporated at a 0.2 A/s rate. See Table 1.

The current-voltage-brightness (J-V-L) characterizations for the OLED devices were performed with a source measurement unit (KEITHLY 238) and a luminescence meter (MINOLTA CS-100A). EL spectra of the OLED devices were collected by a calibrated CCD spectrograph.

TABLE 1
Device Materials

| | | Commercial |
|---|---|---|
| | Name | Name |
| Hole Injection | N1,N1′-([1,1′-biphenyl]-4,4′-diyl)- | |
| Material | bis(N1-(naphthalen-1-yl)-N4,N4- | |
| | diphenylbenzene-1,4-diamine) | |
| Hole Transporting | N4,N4′-di(naphthalen-1-yl)-N4,N4′- | NPB |
| Material | diphenyl-[1,1′-biphenyl]-4,4′-diamine | |
| Fl Blue Host | 9,10-di(naphthalen-2-yl)anthracene | ADN |
| Fl Blue Dopant | (E)-4,4′-(ethene-1,2-diyl)bis(N,N- | DPAVB |
| | diphenylaniline) | |
| Ref ETL | tris(8-hydroxyquinolinato)aluminum | Alq3 |
| Electron Injection | Lithium Quinolate | Liq |
| Material | | |

### Inventive Compounds 1, 2 and 3

Inventive compounds 1, 2 and 3 were each further purified by sublimation, and incorporated into OLED devices for preliminary evaluation against the reference Alq₃. OLED devices were fabricated, as discussed above, on coated glass substrates with multiple organic layers sandwiched between a transparent ITO anode and an aluminum cathode. Table 2 shows the OLED device testing results of inventive compounds 1, 2 and 3 mixed with Liq (50:50), compared to a reference compound. As seen in Table 2, the devices containing the inventive compounds had better (higher) efficiency and better (lower) driving voltage over the device containing the reference compound.

TABLE 2
Device Data

| | Voltage | Luminescence | |
|---|---|---|---|
| | @1000 nit | Efficiency | CIE |
| Device | [V] | @1000 nit [Cd/A] | (X, Y) |
| Alq₃ | 6.6 | 4.0 | 149, 150 |
| Inventive | 4.8 | 6.5 | 149, 149 |
| Compound 1: Liq | | | |
| Inventive | 5.2 | 5.4 | 149, 148 |
| Compound 2: Liq | | | |
| Inventive | 5.0 | 6.0 | 149, 148 |
| Compound 3: Liq | | | |

1. A composition comprising at least one compound selected from Formula 1: 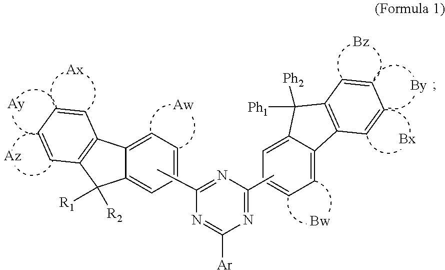
wherein, A is (CH), and x, y, w, z are each independently 0 or 4; and
wherein x=4, and y=0, z=0 or 4, and w=0 or 4, or
wherein y=4, and x=0, z=0, and w=0 or 4, or
wherein z=4, and x=0 or 4, y=0, and w=0 or 4, or
wherein w=4, and x=0, y=0, z=0; and
wherein, B is (CH), and x, y, w, z are each independently 0 or 4; and
wherein x=4, and y=0, z=0 or 4, and w=0 or 4, or
wherein y=4, and x=0, z=0, and w=0 or 4, or
wherein z=4, and x=0 or 4, y=0, and w=0 or 4, or
wherein w=4, and x=0, y=0, z=0; and
wherein R₁ and R₂ are each independently a C1-C20 alkyl or a C6-C30 aryl, each with or without one or more substituents; and
wherein Ph₁ and Ph₂ are each, independently, phenyl or a substituted phenyl; and
wherein Ar is an aryl, a substituted aryl, a heteroaryl, or a substituted heteroaryl; and
wherein Ar comprises less than, or equal to, 16 carbons; and
wherein one or more hydrogens may be optionally substituted with deuterium.

2. The composition of claim 1, wherein Ar comprises from 8 to 16 carbons.

3. The composition of claim 1, wherein the at least one compound is selected from Formula 2:

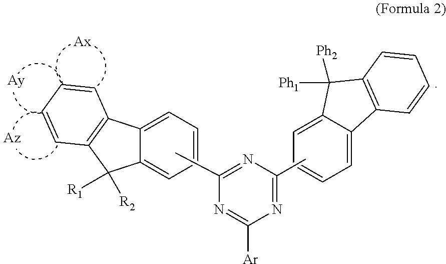

4. The composition of claim 1, wherein Ar is selected from the following groups:

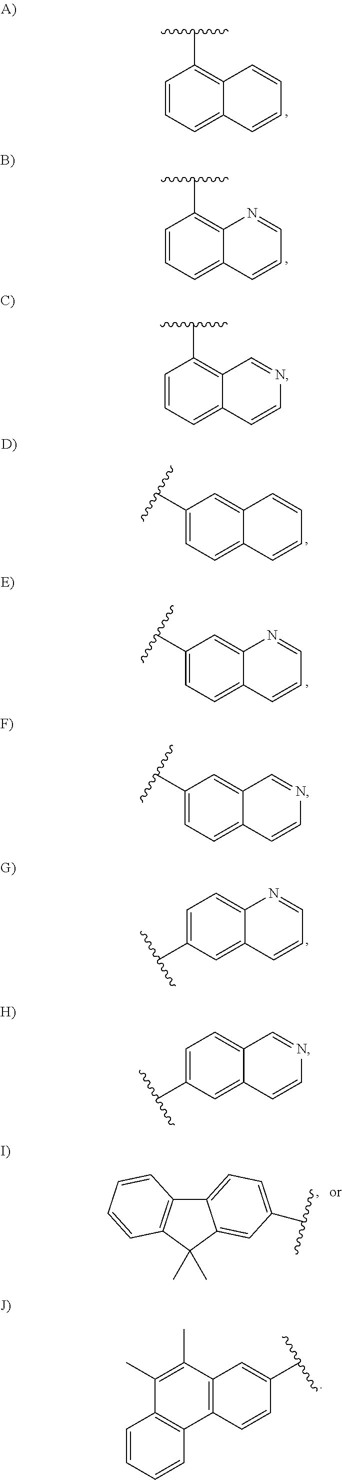

5. The composition of claim 1, wherein the compound has a glass transition temperature (Tg) greater than, or equal to, 150° C.

6. The composition of claim 1, wherein the compound has a molecular weight from 620 g/mole to 1000 g/mole.

7. The composition of claim 1, wherein the compound has a triplet energy from 1.5 eV to 3.0 eV.

8. The composition of claim 1, wherein R₁ and R₂ are each independently a C1-C4 alkyl.

9. The composition of claim 1, wherein the at least one compound is selected from the following:

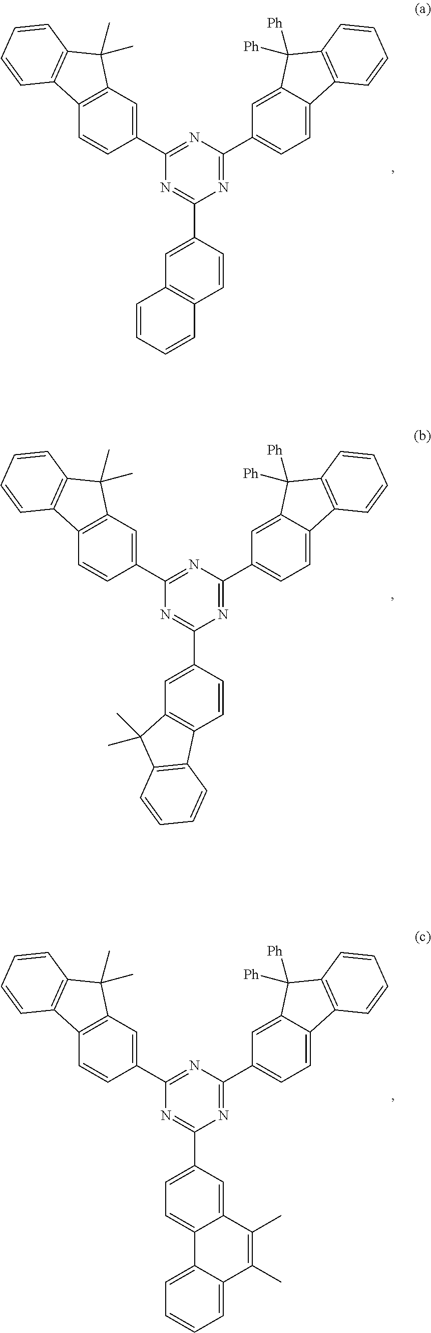 [SMILES: CC1(C)c2ccccc2-c2ccc(-c3nc(-c4ccc5c(c4)C(c4ccccc4)(c4ccccc4)c4ccccc4-5)nc(-c4ccc5ccccc5c4)n3)cc21 ; CC1(C)c2ccccc2-c2ccc(-c3nc(-c4ccc5c(c4)C(C)(C)c4ccccc4-5)nc(-c4ccc5c(c4)C(c4ccccc4)(c4ccccc4)c4ccccc4-5)n3)cc21 ; Cc1c(C)c2cc(-c3nc(-c4ccc5c(c4)C(C)(C)c4ccccc4-5)nc(-c4ccc5c(c4)C(c4ccccc4)(c4ccccc4)c4ccccc4-5)n3)ccc2c2ccccc12]

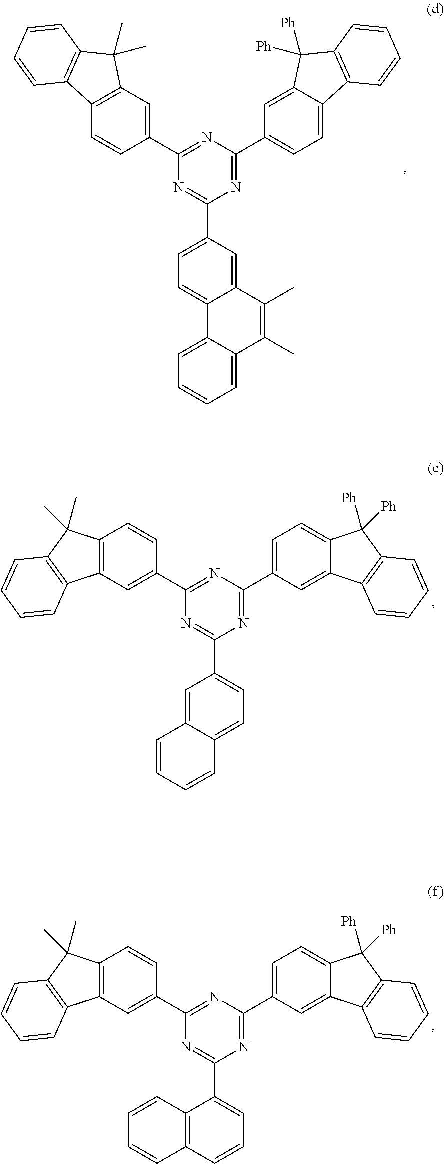 [SMILES: Cc1c(C)c2cc(-c3nc(-c4ccc5c(c4)C(C)(C)c4ccccc4-5)nc(-c4ccc5c(c4)C(c4ccccc4)(c4ccccc4)c4ccccc4-5)n3)ccc2c2ccccc12 ; CC1(C)c2ccccc2-c2cc(-c3nc(-c4ccc5c(c4)-c4ccccc4C5(c4ccccc4)c4ccccc4)nc(-c4ccc5ccccc5c4)n3)ccc21 ; CC1(C)c2ccccc2-c2cc(-c3nc(-c4ccc5c(c4)-c4ccccc4C5(c4ccccc4)c4ccccc4)nc(-c4cccc5ccccc45)n3)ccc21]

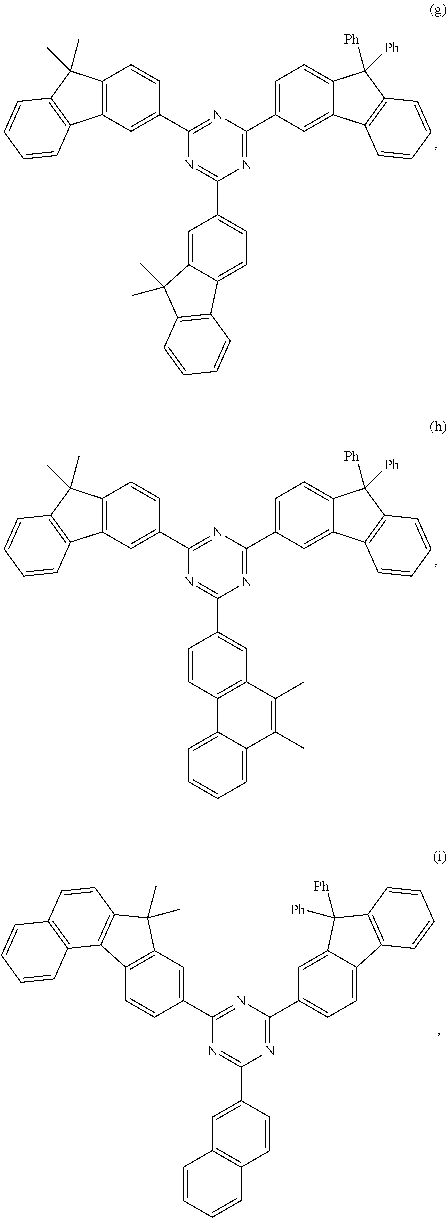 [SMILES: CC1(C)c2ccccc2-c2cc(-c3nc(-c4ccc5c(c4)-c4ccccc4C5(c4ccccc4)c4ccccc4)nc(-c4ccc5c(c4)C(C)(C)c4ccccc4-5)n3)ccc21 ; Cc1c(C)c2cc(-c3nc(-c4ccc5c(c4)-c4ccccc4C5(C)C)nc(-c4ccc5c(c4)-c4ccccc4C5(c4ccccc4)c4ccccc4)n3)ccc2c2ccccc12 ; CC1(C)c2cc(-c3nc(-c4ccc5c(c4)C(c4ccccc4)(c4ccccc4)c4ccccc4-5)nc(-c4ccc5ccccc5c4)n3)ccc2-c2c1ccc1ccccc21]

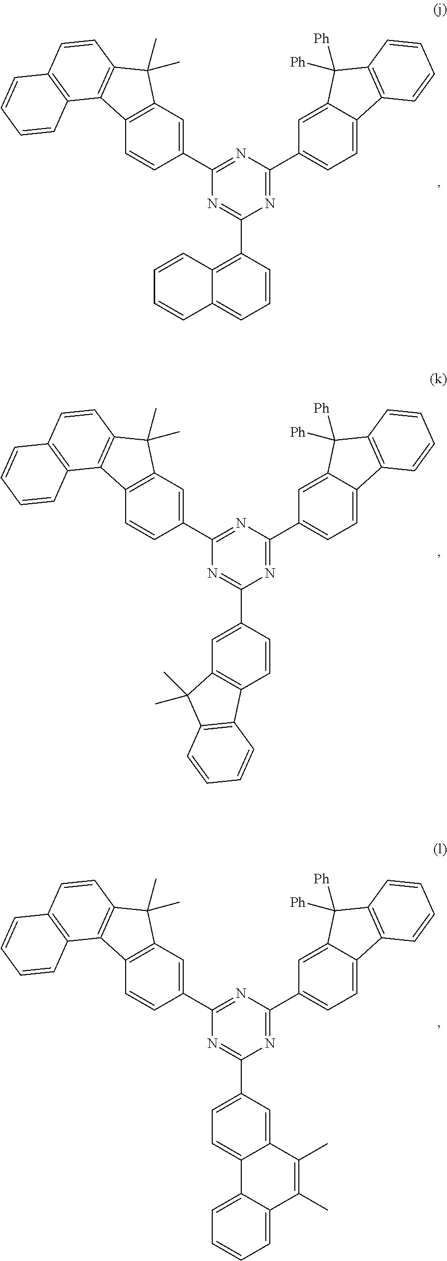 [SMILES: CC1(C)c2cc(-c3nc(-c4ccc5c(c4)C(c4ccccc4)(c4ccccc4)c4ccccc4-5)nc(-c4cccc5ccccc45)n3)ccc2-c2c1ccc1ccccc21 ; CC1(C)c2ccccc2-c2ccc(-c3nc(-c4ccc5c(c4)C(C)(C)c4ccc6ccccc6c4-5)nc(-c4ccc5c(c4)C(c4ccccc4)(c4ccccc4)c4ccccc4-5)n3)cc21 ; Cc1c(C)c2cc(-c3nc(-c4ccc5c(c4)C(C)(C)c4ccc6ccccc6c4-5)nc(-c4ccc5c(c4)C(c4ccccc4)(c4ccccc4)c4ccccc4-5)n3)ccc2c2ccccc12]

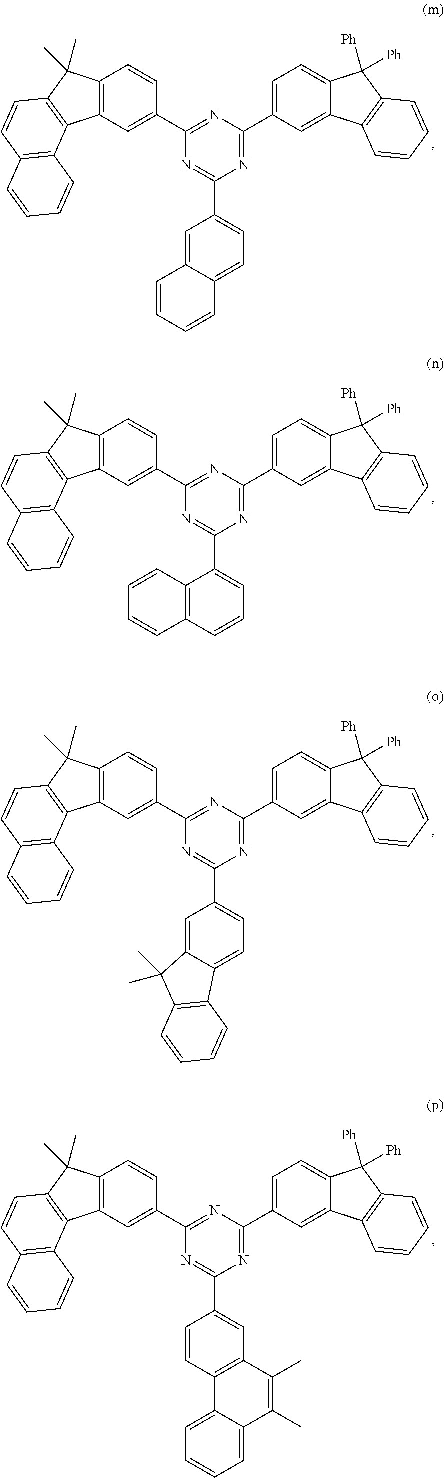 [SMILES: CC1(C)c2ccc(-c3nc(-c4ccc5c(c4)-c4ccccc4C5(c4ccccc4)c4ccccc4)nc(-c4ccc5ccccc5c4)n3)cc2-c2c1ccc1ccccc21 ; CC1(C)c2ccc(-c3nc(-c4ccc5c(c4)-c4ccccc4C5(c4ccccc4)c4ccccc4)nc(-c4cccc5ccccc45)n3)cc2-c2c1ccc1ccccc21 ; CC1(C)c2ccccc2-c2ccc(-c3nc(-c4ccc5c(c4)-c4c(ccc6ccccc46)C5(C)C)nc(-c4ccc5c(c4)-c4ccccc4C5(c4ccccc4)c4ccccc4)n3)cc21 ; Cc1c(C)c2cc(-c3nc(-c4ccc5c(c4)-c4c(ccc6ccccc46)C5(C)C)nc(-c4ccc5c(c4)-c4ccccc4C5(c4ccccc4)c4ccccc4)n3)ccc2c2ccccc12]

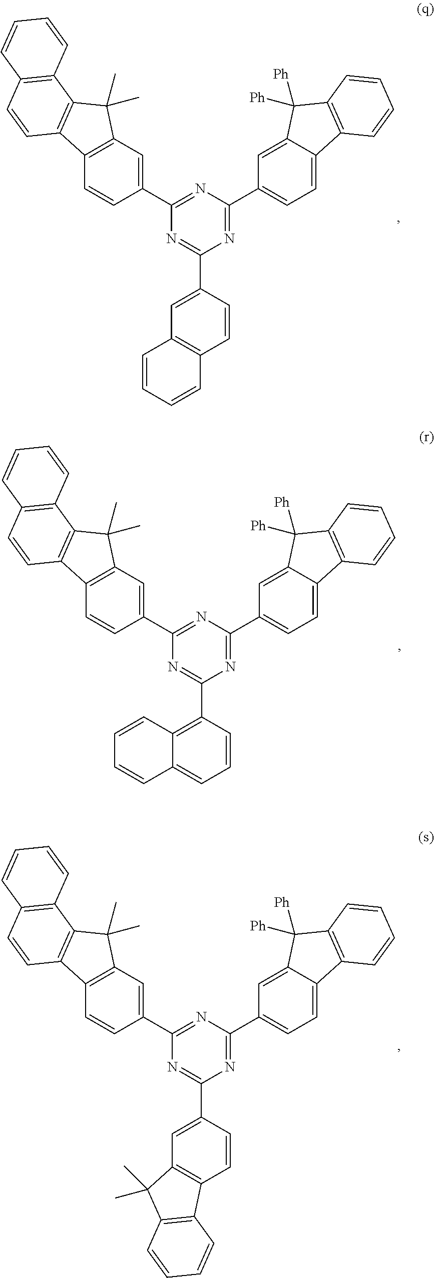 [SMILES: CC1(C)c2cc(-c3nc(-c4ccc5c(c4)C(c4ccccc4)(c4ccccc4)c4ccccc4-5)nc(-c4ccc5ccccc5c4)n3)ccc2-c2ccc3ccccc3c21 ; CC1(C)c2cc(-c3nc(-c4ccc5c(c4)C(c4ccccc4)(c4ccccc4)c4ccccc4-5)nc(-c4cccc5ccccc45)n3)ccc2-c2ccc3ccccc3c21 ; CC1(C)c2ccccc2-c2ccc(-c3nc(-c4ccc5c(c4)C(C)(C)c4c-5ccc5ccccc45)nc(-c4ccc5c(c4)C(c4ccccc4)(c4ccccc4)c4ccccc4-5)n3)cc21]

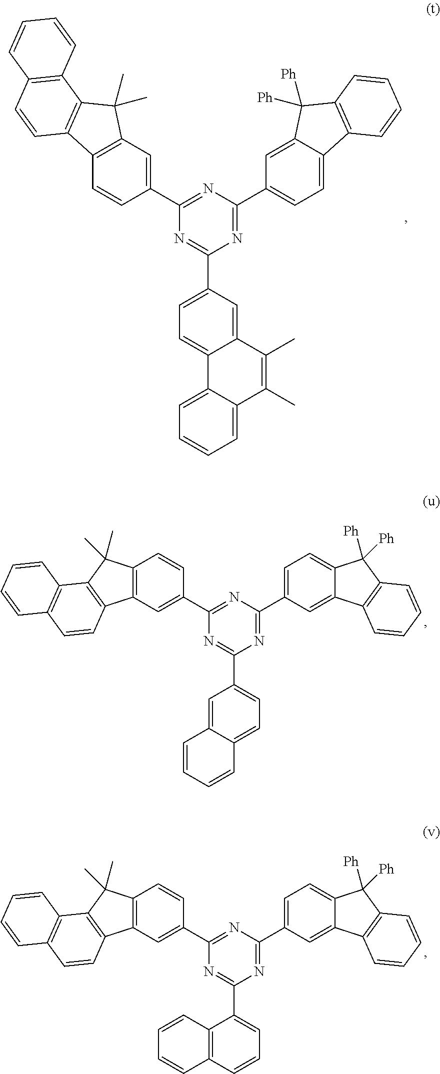 [SMILES: Cc1c(C)c2cc(-c3nc(-c4ccc5c(c4)C(C)(C)c4c-5ccc5ccccc45)nc(-c4ccc5c(c4)C(c4ccccc4)(c4ccccc4)c4ccccc4-5)n3)ccc2c2ccccc12 ; CC1(C)c2ccc(-c3nc(-c4ccc5c(c4)-c4ccccc4C5(c4ccccc4)c4ccccc4)nc(-c4ccc5ccccc5c4)n3)cc2-c2ccc3ccccc3c21 ; CC1(C)c2ccc(-c3nc(-c4ccc5c(c4)-c4ccccc4C5(c4ccccc4)c4ccccc4)nc(-c4cccc5ccccc45)n3)cc2-c2ccc3ccccc3c21]

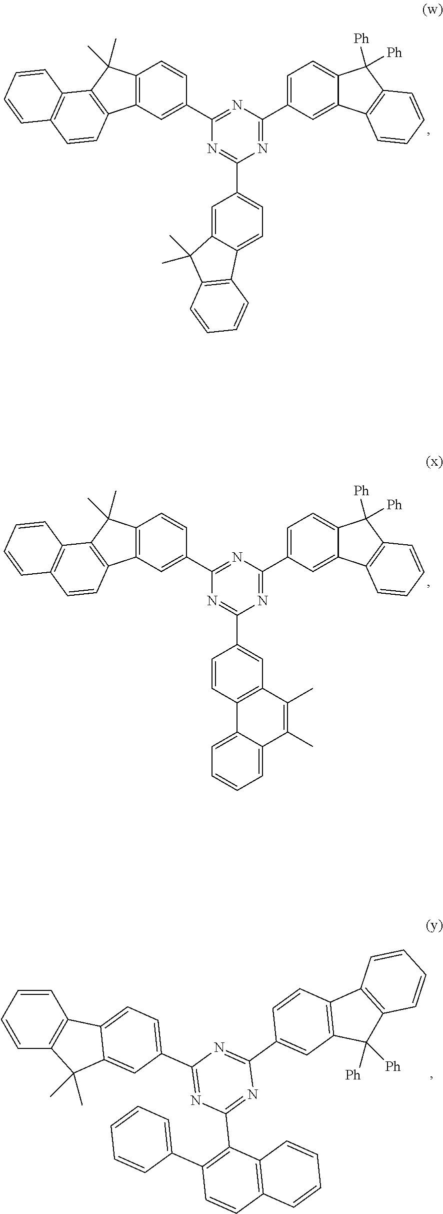 [SMILES: CC1(C)c2ccccc2-c2ccc(-c3nc(-c4ccc5c(c4)-c4ccc6ccccc6c4C5(C)C)nc(-c4ccc5c(c4)-c4ccccc4C5(c4ccccc4)c4ccccc4)n3)cc21 ; Cc1c(C)c2cc(-c3nc(-c4ccc5c(c4)-c4ccc6ccccc6c4C5(C)C)nc(-c4ccc5c(c4)-c4ccccc4C5(c4ccccc4)c4ccccc4)n3)ccc2c2ccccc12 ; CC1(C)c2ccccc2-c2ccc(-c3nc(-c4ccc5c(c4)C(c4ccccc4)(c4ccccc4)c4ccccc4-5)nc(-c4c(-c5ccccc5)ccc5ccccc45)n3)cc21]

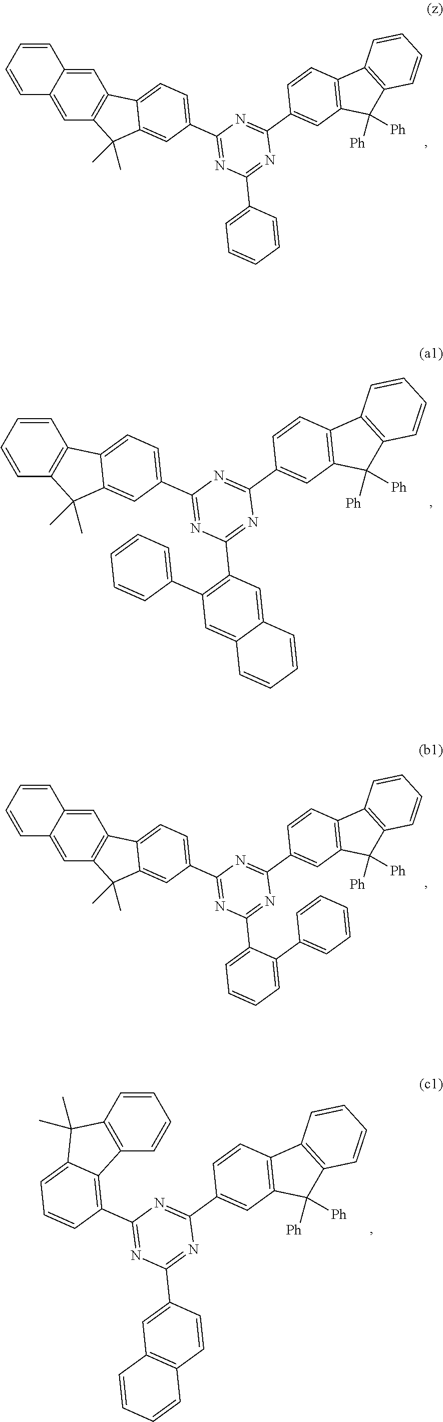 [SMILES: CC1(C)c2cc(-c3nc(-c4ccccc4)nc(-c4ccc5c(c4)C(c4ccccc4)(c4ccccc4)c4ccccc4-5)n3)ccc2-c2cc3ccccc3cc21 ; CC1(C)c2ccccc2-c2ccc(-c3nc(-c4ccc5c(c4)C(c4ccccc4)(c4ccccc4)c4ccccc4-5)nc(-c4cc5ccccc5cc4-c4ccccc4)n3)cc21 ; CC1(C)c2cc(-c3nc(-c4ccc5c(c4)C(c4ccccc4)(c4ccccc4)c4ccccc4-5)nc(-c4ccccc4-c4ccccc4)n3)ccc2-c2cc3ccccc3cc21 ; CC1(C)c2ccccc2-c2c(-c3nc(-c4ccc5c(c4)C(c4ccccc4)(c4ccccc4)c4ccccc4-5)nc(-c4ccc5ccccc5c4)n3)cccc21]

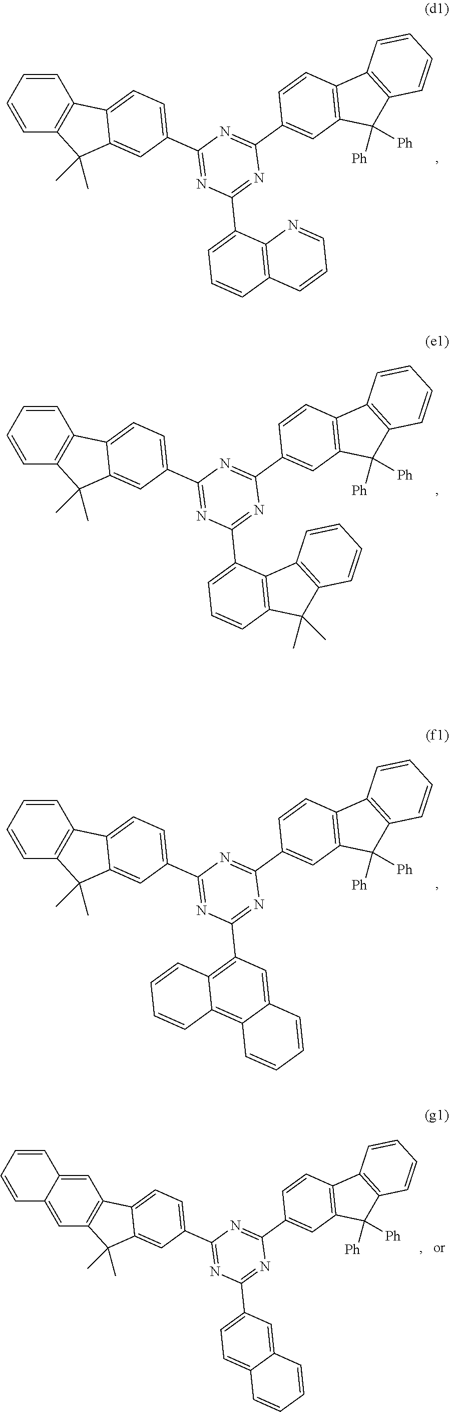 [SMILES: CC1(C)c2ccccc2-c2ccc(-c3nc(-c4ccc5c(c4)C(c4ccccc4)(c4ccccc4)c4ccccc4-5)nc(-c4cccc5cccnc45)n3)cc21 ; CC1(C)c2ccccc2-c2ccc(-c3nc(-c4ccc5c(c4)C(c4ccccc4)(c4ccccc4)c4ccccc4-5)nc(-c4cccc5c4-c4ccccc4C5(C)C)n3)cc21 ; CC1(C)c2ccccc2-c2ccc(-c3nc(-c4ccc5c(c4)C(c4ccccc4)(c4ccccc4)c4ccccc4-5)nc(-c4cc5ccccc5c5ccccc45)n3)cc21 ; CC1(C)c2cc(-c3nc(-c4ccc5c(c4)C(c4ccccc4)(c4ccccc4)c4ccccc4-5)nc(-c4ccc5ccccc5c4)n3)ccc2-c2cc3ccccc3cc21]

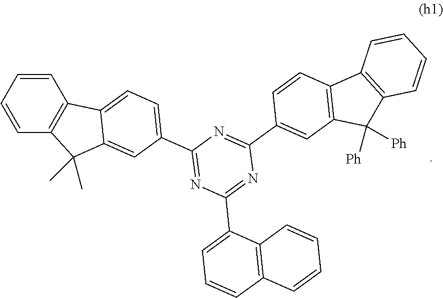 [SMILES: CC1(C)c2ccccc2-c2ccc(-c3nc(-c4ccc5c(c4)C(c4ccccc4)(c4ccccc4)c4ccccc4-5)nc(-c4cccc5ccccc45)n3)cc21]

10. A film comprising at least one Layer A formed from the composition of claim 1.

11. The film of claim 10, further comprising another Layer B, which is formed from a Composition B comprising at least one HTL compound.

12. The film of claim 10, wherein Layer A is not adjacent to Layer B.

13. An article comprising at least one component formed from the composition of claim 1.

14. The article of claim 13, wherein the article is an electroluminescent device.
